(19) United States
(12) Patent Application Publication
(10) Pub. No.: US 2024/0342271 A1
(43) Pub. Date: Oct. 17, 2024

(54) SYNERGISM OF IMMUNOGENICITY VIA COMBINED PARENTERAL AND MUCOSAL IMMUNIZATION AGAINST COVID-19

(71) Applicant: BHARAT BIOTECH INTERNATIONAL LIMITED, Hyderabad (IN)

(72) Inventors: Raches Ella, Hyderabad (IN); Krishna Mohan VADREVU, Hyderabad (IN); Raju SUNAGAR, Hyderabad (IN); Krishna Murthy ELLA, Hyderabad (IN)

(73) Assignee: BHARAT BIOTECH INTERNATIONAL LIMITED, Hyderabad (IN)

(21) Appl. No.: 18/294,775

(22) PCT Filed: Aug. 2, 2022
(86) PCT No.: PCT/IN2022/050694

§ 371 Date: Feb. 2, 2024

(30) Foreign Application Priority Data
Aug. 3, 2021 (IN) 202141035009
Oct. 7, 2021 (IN) 202141045615

(51) Int. Cl. A61K 39/215; A61K 39/00; A61P 37/04

(52) U.S. Cl. CPC A61K 39/215; A61P 37/04; A61K 2039/5252; A61K 2039/5256; A61K 2039/53; A61K 2039/54; A61K 2039/542; A61K 2039/543; A61K 2039/545

(57) ABSTRACT

The present invention discloses a system and method of generating robust immune response in mammals against SARS-CoV-2 antigen by administering two or more doses of same or different COVID-19 vaccines through same or different routes, wherein at least one vaccine is selected from a primary series of vaccines and at least one vaccine is selected from a secondary series of vaccines and wherein vaccines of primary and secondary series are administered through homologous or heterologous routes. The homologous route of administration comprises administering primary and secondary series of vaccines through same route. The heterologous route of administration comprises administering primary and secondary series vaccines through different routes. The system and method of the invention induces superior cross protection against SARS-CoV-2 variants including against Delta and Omicron variants.

### RELATED PATENT APPLICATIONS

This application is a cognate application of the Provisional Specifications filed at the Indian Patent Office under Indian Patent Application No. 202141035009 filed on Aug. 3, 2021, and Indian Patent Application No. 202141045615 filed on Oct. 7, 2021; the disclosures of which are incorporated herein by reference.

### FIELD OF INVENTION

The present invention relates to a homologous or heterologous vaccination approach to induce robust immune response to SARS-CoV-2 infection in mammals. More particularly, present invention relates to synergism of immunogenicity via combined parental and mucosal immunization against COVID-19. The invention further relates to a method of generating robust immune response in mammals against SARS-CoV-2 antigen by administering two or more doses of same or different COVID-19 vaccines, wherein said vaccines are administered as one or more doses of primary series of vaccines and one or more doses of secondary series of vaccines wherein the primary and secondary series of vaccines are administered through heterologous or homologous routes. The invention also relates to combined parental-mucosal immunization to induce mucosal and systemic immune response against SARS CoV-2 infections or COVID-19.

### BACKGROUND OF THE INVENTION

The COVID-19 pandemic, that started in Wuhan, China in the end of 2019, is caused by the infection and transmission of “severe acute respiratory syndrome coronavirus 2” (SARS-CoV-2). The disease is an acute respiratory illness ranging in severity from mild to severe, with death in some cases. COVID-19 is highly contagious spreading rapidly around the world, it was observed that the transmission of the SARS-CoV-2 happen before the appearance of the symptoms.

Vaccines could play an important role in providing immunity, preventing severe disease, and reducing the ongoing health crisis. To date, more than 200 SARS-CoV2 vaccine candidates are being developed to prevent COVID-19. Most of them have reached late-stage clinical trials with encouraging immunogenicity and efficacy results (COVID-19 vaccine tracker and landscape, WHO; COVID-19 Treatment and Vaccine Tracker, Milken Institute). In control of the circulating SARS-CoV2 variants of concern, homologous parenteral prime followed by boost vaccine schedules are reporting decreased immunogenicity and efficacy over the course of time. Therefore, there is significant global interest in exploring heterologous prime-boost COVID-19 vaccination to make vaccination programmes more flexible and reliable in response to fluctuations in demand and supply.

The combination of broad epitope protection with heightened cell mediated responses may serve to augment the efficacy of heterologous prime boost schedule.

The effective vaccination strategies include and not limited to
i) Different vaccines—Heterologous route of administration
ii) Same vaccine—Heterologous route of administration
iii) Different vaccines—Homologous route of administration

So far, all the approved vaccines for SARS-CoV-2 are developed based on intramuscular (IM) route of administration. Most of the developed SARS-CoV-2 vaccines are designed for intramuscular (IM) administration to induce humoral and cell mediated immune responses, thereby to prevent SARS CoV-2 infection. More or less, all developed vaccines showed good efficacy in humans in a range of 70-95% depending on the type of vaccine.

However, little protection is provided against viral replication in the upper airways due to the lack of IgA response. These IM COVID-19 vaccines are designed to elicit robust systemic immunity but induce limited mucosal immunity that is critical for blocking SARS-CoV-2 infection and transmission, leading to breakthrough infection in fully vaccinated individuals (Neeltje van Doremalen et. al., ChAdOx1 nCoV-19 vaccine prevents SARS-CoV-2 pneumonia in rhesus macaques, Nature 586, 578-582, doi:10.1038/s41586-020-2608-y (2020).)

Presently available licensed COVID-19 vaccines are administered by injection, these IM injected COVID-19 vaccines predominantly elicits an IgG and prevents viremia with limited mucosal protection.

It is known that the transmission of the SARS-CoV-2 virus occurs via respiratory droplets from cough and sneezes. It has been shown that the nasal epithelium expresses more ACE2 receptors. Once virus enters the airways, spike (S) protein of SARS-CoV-2 interacts with ACE 2 receptors. It is expected that the proteolytic cleavage of Spike protein, mediated by the cellular protease TMPRSS2, facilitates SARS-CoV-2 infection by facilitating the replication of the virions. Furthermore, the tendency of high mutation rate of SARS-CoV-2 due to the presence of RdRp (RNA-dependent RNA polymerases) of the virus, may lead to the generation of virus variants, also known as Variants of Concern (VOC).

SARS-CoV-2 virus enters the humans mainly through the ACE2⁺TMPRSS2⁺ nasal epithelial cells. Based on the available literature, the initial defence mechanism against pathogen starts at Nasopharynx-Associated Lymphoid Tissue (NALT) system and the central role of NALT appears to be the key factor in the regulation of the immune response. Hence, subsequent immune responses occurred following the initial immune response at nasal site could help in preventing the virus transmission. Hence, the nasal route of delivery attracted more attention, in the vaccine development research against SARS CoV-2.

Preclinical animal studies in various lab animals' species demonstrated that COVID-19 vaccines that elicit IgA mediated mucosal immunity could be the most critical defence mechanism against SARS-CoV-2 and may reduce viral shedding and may block infection or transmission.

However, the Phase 1 clinical trial with intranasal vaccination of recombinant human Ad5 expressing SARS-CoV-2 spike (AdCOVID) showed substantially lower magnitude of the immune response with high percent of non-responders. Suggesting that, prior immunity in humans may be important for a robust immune response to intranasal dosing.

Moreover, widespread of SARS CoV-2 virus and emergence of new variants due to occurrence of mutations pose serious threat to global health. Hence, there is a great challenge to develop a vaccine that induce both mucosal and robust systemic immune response that can prevent virus transmission.

Effective vaccination strategies need not be restricted to a single route of administration of vaccine, several vaccine studies have demonstrated that memory cells primed by parenteral vaccination can be “pulled” into mucosal sites by successive mucosal vaccination (Frances E. Lund et. al., Scent of a vaccine, Science 373, 397-399, doi:10.1126/science.abg9857 (2021); Qian He, Lang Jiang et al., A Systemic Prime-Intrarectal Pull Strategy Raises Rectum-Resident CD8+ T Cells for Effective Protection in a Murine Model of LM-OVA Infection, Front Immunol 11, 571248, doi:10.3389/fimmu.2020.571248 (2020); David L Bernstein et. al., Successful application of prime and pull strategy for a therapeutic HSV vaccine, NPJ Vaccines 4, 33, doi:10.1038/s41541-019-0129-1 (2019)).

In addition to stand-alone intranasal mucosal vaccine approaches, parenteral prime and mucosal boost strategies offer promise.

With this background, current invention describes the methods for heterologous vaccination for generating a robust, immune response in mammals against a SARS-CoV-2 infection to prevent COVID-19. A parenteral prime followed by a mucosal booster vaccination or vice versa, induces qualitatively and quantitatively better immune response, including significant reduction or prevention of in viral replication in the upper and lower respiratory tracts in mammals and it is also suitable for immunizing human subjects. Present invention provides the use of the heterologous vaccination approach to induce broad immune response against SARS CoV-2 infection(s), by administering vaccine via different routes using various adjuvant formulations or various vaccine platforms.

### OBJECTS OF THE INVENTION

One object of the invention is to provide a method of vaccination for prevention of SARS-CoV-2 infections as well as infections caused by other coronaviruses such as SARS-CoV and MERS-CoV.

Another object of the invention is to provide a method of generating a robust immune response in mammals against COVID-19 by utilizing homologous or heterologous vaccination regimen.

In one object present invention provides one or more vaccines selected from a primary series of vaccines and one or more vaccines selected from a secondary series of vaccines.

Another object of the invention is to provide a method of vaccination wherein vaccines selected from primary and secondary series are administered through homologous or heterologous routes.

A further object of the invention is to provide synergism of immunogenicity via combined parental and mucosal immunization against COVID-19.

Another object of the invention is to provide heterologous immunization to induce qualitatively and quantitatively better immune response, including significant reduction or prevention of in viral replication in the upper and lower respiratory tracts in mammals, also suitable for immunizing human subjects.

Another object of present invention is to provide an immunogenic system for generating robust immune response in mammals against SARS CoV-2 infection.

A further object of the invention is to provide a combined parental-mucosal immunization to induce mucosal and systemic immune response against SARS CoV-2 infections or COVID-19.

Another object of the invention is to provide an immunogenic system for heterologous vaccination against SARS CoV-2 infections wherein same/different vaccines are administered through homologous or heterologous route.

Another object of the invention is to provide a method of generating increased IgG and IgA antibody response.

Another object of the invention is to provide a method of generating increased neutralizing antibody response.

A further object of the present invention is to assess ability of new adjuvant formulations to induce both humoral and cell mediated T cell responses and mucosal responses (IgA titers).

Yet another objective of the invention is to provide the method of generating superior cross protection against SARS-CoV-2 variants such as Delta or omicron compared to homologous regime.

### SUMMARY OF THE INVENTION

The present invention is related a homologous or heterologous vaccination approach to induce robust immune response to SARS-CoV-2 infection in mammals. More particularly, present invention relates to synergism of immunogenicity via combined parental and mucosal immunization against COVID-19.

Present invention discloses and describes the method of generating a robust immune response in mammals, also suitable for humans against COVID-19 by utilizing homologous or heterologous vaccination regimen.

Further the invention is related with the method of generating a robust immune response in mammals, against a SARS-CoV-2 antigen by administering at least two doses of COVID-19 vaccines, wherein the said doses are administered in heterologous (parenteral and mucosal) or similar routes.

The present invention further discloses subject administering a one composition through intramuscular or intradermal route, whereas another immunogenic composition administered via intranasal or oral or mucosal routes or the other way around.

In one aspect the present invention provides a method of generating robust immune response in mammals against SARS-CoV-2 antigen by administering two or more doses of same or different COVID-19 vaccines through same or different routes, wherein at least one vaccine is selected from a primary series of vaccines and at least one vaccine is selected from a secondary series of vaccines and wherein vaccines of primary and secondary series are administered through homologous or heterologous routes.

In the said method the homologous route of administration comprises administering primary and secondary series of vaccines through same route.

In homologous route all vaccines of primary and secondary series are administered through the same route selected from intramuscular, intradermal, intranasal, oral and mucosal route.

The heterologous route of administration comprises administering primary and secondary series vaccines through different routes.

In heterologous route of administration at least two vaccines from primary and secondary series are administered through different routes.

In heterologous route at least one vaccine from primary or secondary series is administered through intramuscular or intradermal route and at least one vaccine from primary or secondary series is administered through intranasal, oral or mucosal route.

In one embodiment, both primary and secondary series of vaccines comprise of killed-inactivated SARS-CoV-2 whole-virion vaccine.

In another embodiment, both primary and secondary series of vaccines comprise of recombinant adenovectored SARS-CoV-2 virus vaccine.

In one embodiment, the primary series of vaccine comprises at least one killed-inactivated SARS-CoV-2 whole-virion vaccine and secondary series of vaccine comprises at least one recombinant adenovectored SARS-CoV-2 virus vaccine.

In one embodiment, the primary series of vaccine comprises at least one recombinant adenovectored SARS-CoV-2 virus vaccine and secondary series of vaccine comprises at least one killed-inactivated SARS-CoV-2 whole-virion vaccine.

In the said method the recombinant adenovectored SARS-CoV-2 virus vaccine is selected from recombinant chimpanzee adenovirus (ChAd-SARS-CoV-2-S), recombinant human adenovirus (hAd-SARS-CoV-2-S), rabies vectored vaccine, respiratory syncytial virus vectored vaccine (RSV), influenza virus (both A and B strains) or other respiratory virus vectored vaccine containing gene segment for full length or partial spike protein or immunogenic part thereof from SARS-CoV-2 virus.

In one embodiment, the recombinant adenovectored SARS-CoV-2 virus vaccine is a recombinant chimpanzee adenovirus containing gene segment for full length or partial genome encoding complete or partial spike protein or immunogenic part thereof from SARS-CoV-2 virus (ChAd-SARS-CoV-2-S).

In one embodiment, the recombinant adenovectored SARS-CoV-2 virus vaccine is a recombinant human adenovirus (hAd-SARS-CoV-2-S) containing nucleic acid encoding full length codon optimized spike protein of SARS-CoV-2 (rAd-nCoV-Spike).

In some embodiment, both primary and secondary series of vaccines comprise recombinant chimpanzee adenovirus (ChAd-SARS-CoV-2-S) with the genome encoding complete or partial spike protein or immunogenic part thereof from SARS-CoV-2.

In some embodiment, both primary and secondary series of vaccines comprise of recombinant human adenovirus (hAd-SARS-CoV-2-S) with nucleic acid encoding full length codon optimized spike protein of SARS-CoV-2 (rAd-nCoV-Spike).

In one embodiment, the primary series of vaccine comprises of killed-inactivated SARS-CoV-2 whole-virion vaccine and the secondary series of vaccine comprises recombinant chimpanzee adenovirus (ChAd-SARS-CoV-2-S) with the genome encoding complete or partial spike protein or immunogenic part thereof from SARS-CoV-2.

The vaccines of primary and secondary series may be selected from—

| | Primary series | | Secondary series | |
|---|---|---|---|---|
| 1. | recombinant | recombinant | recombinant | recombinant |
| | adenovectored | adenovectored | adenovectored | adenovectored |
| | SARS-CoV-2 virus | SARS-CoV-2 virus | SARS-CoV-2 virus | SARS-CoV-2 |
| | vaccine | vaccine | vaccine | virus vaccine |
| 2. | killed-inactivated | killed-inactivated | killed-inactivated | killed-inactivated |
| | SARS-CoV-2 | SARS-CoV-2 | SARS-CoV-2 | SARS-CoV-2 |
| | whole-virion | whole-virion | whole-virion | whole-virion |
| | vaccine | vaccine | vaccine | vaccine |
| 3. | killed-inactivated | killed-inactivated | recombinant | recombinant |
| | SARS-CoV-2 | SARS-CoV-2 | adenovectored | adenovectored |
| | whole-virion | whole-virion | SARS-CoV-2 virus | SARS-CoV-2 |
| | vaccine | vaccine | vaccine. | virus vaccine. |
| 4. | killed-inactivated | recombinant | killed-inactivated | recombinant |
| | SARS-CoV-2 | adenovectored | SARS-CoV-2 | adenovectored |
| | whole-virion | SARS-CoV-2 virus | whole-virion | SARS-CoV-2 |
| | vaccine | vaccine. | vaccine | virus vaccine. |
| 5. | recombinant human | recombinant human | recombinant | recombinant |
| | adenovirus (hAd- | adenovirus (hAd- | human adenovirus | human adenovirus |
| | SARS-CoV-2-S) | SARS-CoV-2-S) | (hAd-SARS-CoV- | (hAd-SARS- |
| | | | 2-S) | CoV-2-S) |
| 6. | recombinant | recombinant | recombinant | recombinant |
| | chimpanzee | chimpanzee | chimpanzee | chimpanzee |
| | adenovirus (ChAd- | adenovirus (ChAd- | adenovirus (ChAd- | adenovirus |
| | SARS-CoV-2-S) | SARS-CoV-2-S) | SARS-CoV-2-S) | (ChAd-SARS- |
| | | | | CoV-2-S) |
| 7. | recombinant human | recombinant | recombinant | recombinant |
| | adenovirus (hAd- | chimpanzee | human adenovirus | chimpanzee |
| | SARS-CoV-2-S) | adenovirus (ChAd- | (hAd-SARS-CoV- | adenovirus |
| | | SARS-CoV-2-S) | 2-S) | (ChAd-SARS- |
| | | | | CoV-2-S) |
| 8. | recombinant human | recombinant human | recombinant | recombinant |
| | adenovirus (hAd- | adenovirus (hAd- | chimpanzee | chimpanzee |
| | SARS-CoV-2-S) | SARS-CoV-2-S) | adenovirus (ChAd- | adenovirus |
| | | | SARS-CoV-2-S) | (ChAd-SARS- |
| | | | | CoV-2-S) |
| 9. | recombinant | recombinant | recombinant | recombinant |
| | chimpanzee | chimpanzee | human adenovirus | human adenovirus |
| | adenovirus (ChAd- | adenovirus (ChAd- | (hAd-SARS-CoV- | (hAd-SARS- |
| | SARS-CoV-2-S) | SARS-CoV-2-S) | 2-S) | CoV-2-S) |
| 10 | killed-inactivated | killed-inactivated | recombinant | recombinant |
| | SARS-CoV-2 | SARS-CoV-2 | human adenovirus | human adenovirus |
| | whole-virion | whole-virion | (hAd-SARS-CoV- | (hAd-SARS- |
| | vaccine | vaccine | 2-S) | CoV-2-S) |
| 11 | killed-inactivated | killed-inactivated | recombinant | recombinant |
| | SARS-CoV-2 | SARS-CoV-2 | chimpanzee | chimpanzee |
| | whole-virion | whole-virion | adenovirus (ChAd- | adenovirus |
| | vaccine | vaccine | SARS-CoV-2-S) | (ChAd-SARS- |
| | | | | CoV-2-S) |
| 12 | recombinant human | recombinant human | killed-inactivated | recombinant |
| | adenovirus (hAd- | adenovirus (hAd- | SARS-CoV-2 | chimpanzee |
| | SARS-CoV-2-S) | SARS-CoV-2-S) | whole-virion | adenovirus |
| | | | vaccine | (ChAd-SARS- |
| | | | | CoV-2-S) |
| 13 | killed-inactivated | recombinant | killed-inactivated | recombinant |
| | SARS-CoV-2 | chimpanzee | SARS-CoV-2 | chimpanzee |
| | whole-virion | adenovirus (ChAd- | whole-virion | adenovirus |
| | vaccine | SARS-CoV-2-S) | vaccine | (ChAd-SARS- |
| | | | | CoV-2-S) |
| 14 | killed-inactivated | killed-inactivated | recombinant | recombinant |
| | SARS-CoV-2 | SARS-CoV-2 | chimpanzee | chimpanzee |
| | whole-virion | whole-virion | adenovirus (ChAd- | adenovirus |
| | vaccine | vaccine | SARS-CoV-2-S) | (ChAd-SARS- |
| | | | | CoV-2-S) |
| 15 | killed-inactivated | recombinant human | killed-inactivated | recombinant |
| | SARS-CoV-2 | adenovirus (hAd- | SARS-CoV-2 | chimpanzee |
| | whole-virion | SARS-CoV-2-S) | whole-virion | adenovirus |
| | vaccine | | vaccine | (ChAd-SARS- |
| | | | | CoV-2-S) |

In one embodiment, at least one vaccine of primary series is administered through intramuscular or intradermal route and at least one vaccine of secondary series is administered through intranasal or oral or mucosal route.

In one embodiment, at least one vaccine of primary series is administered through intranasal or oral or mucosal route and at least one vaccine of secondary series is administered through intramuscular or intradermal route.

In one embodiment, the primary and secondary series vaccines are administered through heterologous routes.

In one embodiment, the primary and secondary series vaccines are administered through homologous route.

In some embodiment, two vaccines of primary series and one vaccine of secondary series is administered through heterologous route.

In some embodiment, one vaccine of primary series and two vaccines are secondary series are administered through heterologous routes.

In a general embodiment, the second vaccine of primary series is administered between 4-10 weeks after the first vaccine of primary series.

In a general embodiment, the first vaccine of secondary series is administered no less than about 10 weeks after the second vaccine of primary series.

The dose concentration of recombinant chimpanzee adenovirus vaccine (ChAd-SARS-CoV-2-S) is between 10¹⁰ VP/dose-10¹² VP/dose in 0.2-0.5 ml dose volume.

The dose concentration of (ChAd-SARS-CoV-2-S) vaccine is 1×10¹¹ VP/dose at 0.5 mL volume.

The dose concentration of killed-inactivated SARS-CoV-2 whole-virion vaccine is 6 ug/dose in 0.5 mL volume.

In the present method the primary or secondary series vaccines optionally comprise nucleic acid- (DNA or mRNA) or protein [subunit (spike, RBD, S1)] based COVID-19 vaccines.

The present method induces superior cross protection against SARS-CoV-2 variants including against Delta and Omicron variants.

In the said method the heterologous route of administration of primary and secondary series vaccines elicits increased IgG and IgA antibody response than homologous route.

The heterologous route of administration of primary and secondary series vaccine elicits increased neutralizing antibody response compared to homologous regimen.

The heterologous route of administration of primary and secondary series vaccines elicits increased mucosal T cell response than homologous route.

In another aspect present invention provides a method of generating robust immune response in mammals against SARS-CoV-2 antigen by administering one or more vaccines of killed-inactivated SARS-CoV-2 whole-virion vaccine through intramuscular or intradermal route and a one or more vaccines of recombinant chimpanzee adenovirus (ChAd-SARS-CoV-2-S) vaccine through intranasal or oral or mucosal route.

The recombinant chimpanzee adenovirus contains gene segment encoding complete or partial spike protein or immunogenic part thereof from SARS-CoV-2 virus (ChAd-SARS-CoV-2-S)

In the present method the first vaccine of secondary series is administered between 4-10 weeks after the last vaccine of primary series.

In another aspect present invention provides a method of generating robust immune response in mammals against SARS-CoV-2 antigen by administering one or more recombinant chimpanzee adenovirus (ChAd-SARS-CoV-2-S) vaccine through intranasal or oral or mucosal route and one or more killed-inactivated SARS-CoV-2 whole-virion vaccine through intramuscular or intradermal route.

The recombinant chimpanzee adenovirus contains gene segment encoding complete or partial spike protein or immunogenic part thereof from SARS-CoV-2 virus (ChAd-SARS-CoV-2-S)

In the present method the first vaccine of secondary series is administered between 4-10 weeks after the last vaccine of primary series.

In another aspect present invention provides a method of generating robust immune response in mammals against SARS-CoV-2 antigen by administering one or more recombinant human adenovirus (hAd-SARS-CoV-2-S) vaccine through intramuscular or intradermal route and one or recombinant human adenovirus (hAd-SARS-CoV-2-S) vaccine through intranasal or oral or mucosal route.

In another aspect present invention provides a method of generating robust immune response in mammals against SARS-CoV-2 antigen by administering one or more recombinant human adenovirus (hAd-SARS-CoV-2-S) vaccine through intranasal or oral or mucosal route and one or more recombinant human adenovirus (hAd-SARS-CoV-2-S) vaccine through intramuscular or intradermal route.

In another aspect present invention provides a method of generating robust immune response in mammals against SARS-CoV-2 antigen by administering one or more recombinant human adenovirus (hAd-SARS-CoV-2-S) vaccine through intradermal or intramuscular route and one or more recombinant chimpanzee adenovirus (ChAd-SARS-CoV-2-S) vaccine through intranasal or oral or mucosal route.

Yet in another aspect present invention provides an immunogenic system for generating robust immune response in mammals against SARS CoV-2 infection through homologous or heterologous administration of primary and secondary series vaccines, wherein the primary and secondary series vaccines comprise:
a. One or more killed-inactivated SARS-CoV-2 whole-virion vaccine; and
b. One or more recombinant adenovectored SARS-CoV-2 virus vaccine.

In the said system the recombinant adenovectored SARS-CoV-2 virus vaccine may be selected from recombinant chimpanzee adenovirus (ChAd-SARS-CoV-2-S), recombinant human adenovirus (hAd-SARS-CoV-2-S), rabies vectored vaccine, respiratory syncytial virus vectored vaccine (RSV), influenza virus (both A and B strains) and other respiratory virus vectored vaccine containing gene segment for full length or partial spike protein or immunogenic part thereof from SARS-CoV-2 virus.

The recombinant adenovectored SARS-CoV-2 vaccine is recombinant chimpanzee adenovirus (ChAd-SARS-CoV-2-S) vaccine.

The recombinant chimpanzee adenovirus (ChAd-SARS-CoV-2-S) vaccine contains genome encoding complete or partial spike protein or immunogenic part thereof from SARS-CoV-2.

The recombinant adenovectored SARS-CoV-2 vaccine is recombinant human adenovirus (hAd-SARS-CoV-2-S).

The recombinant adenovectored SARS-CoV-2 vaccine is recombinant human adenovirus (hAd-SARS-CoV-2-S) containing nucleic acid encoding full length codon optimized spike protein of SARS-CoV-2 (rAd-nCoV-Spike).

The dose concentration of killed-inactivated SARS-CoV-2 whole-virion vaccine is in the range of 5 μg/dose-7 μg/dose in 0.5 mL volume.

Preferably, the dose concentration of killed-inactivated SARS-CoV-2 whole-virion vaccine is 6 μg/dose in 0.5 mL volume.

The dose concentration of recombinant adenovectored SARS-CoV-2 virus vaccine is in the range of 10¹⁰ VP/dose-10¹² VP/dose in 0.2-0.5 ml dose.

Preferably, the dose concentration of recombinant adenovectored SARS-CoV-2 virus vaccine is 1×10¹¹ VP/dose at 0.5 mL volume.

In the said system the killed-inactivated SARS-CoV-2 whole-virion vaccine is an injection for intramuscular or intradermal administration.

In the said system the recombinant adenovectored SARS-CoV-2 vaccine is a nasal vaccine for intranasal or oral or mucosal administration.

In the said system killed-inactivated SARS-CoV-2 whole-virion vaccine is a nasal vaccine for intranasal or oral or mucosal administration.

In the said system the recombinant adenovectored SARS-CoV-2 vaccine is an injection for intramuscular or intradermal administration.

The present system optionally comprises nucleic acid vaccines such as DNA or mRNA; or protein such as subunit spike, RBD or S1.

In the said system the vaccines are formulated with an adjuvant.

Further in the said system the vaccines are formulated without any adjuvant. The vaccines are stable at 2-8° C.

The heterologous administration of the vaccines elicits increased IgG and IgA antibody response than homologous route.

The heterologous administration of the vaccines elicits increased neutralizing antibody response compared to homologous regimen.

The heterologous administration of the vaccines elicits increased mucosal T cell response than homologous route.

The present system induces enhanced cross protection against all known COVID-19 variants including Delta and Omicron.

According to present system, the primary and secondary series vaccines may be selected from—

| | Primary series | | Secondary series | |
|---|---|---|---|---|
| 1 | recombinant | recombinant | recombinant | recombinant |
| | adenovectored | adenovectored | adenovectored | adenovectored |
| | SARS-CoV-2 virus | SARS-CoV-2 virus | SARS-CoV-2 virus | SARS-CoV-2 virus |
| | vaccine. | vaccine. | vaccine. | vaccine. |
| 2 | killed-inactivated | killed-inactivated | killed-inactivated | killed-inactivated |
| | SARS-CoV-2 whole- | SARS-CoV-2 whole- | SARS-CoV-2 whole- | SARS-CoV-2 whole- |
| | virion vaccine | virion vaccine | virion vaccine | virion vaccine |
| 3 | killed-inactivated | killed-inactivated | recombinant | recombinant |
| | SARS-CoV-2 whole- | SARS-CoV-2 whole- | adenovectored | adenovectored |
| | virion vaccine | virion vaccine | SARS-CoV-2 virus | SARS-CoV-2 virus |
| | | | vaccine. | vaccine. |
| 4 | killed-inactivated | recombinant | killed-inactivated | recombinant |
| | SARS-CoV-2 whole- | adenovectored | SARS-CoV-2 whole- | adenovectored |
| | virion vaccine | SARS-CoV-2 virus | virion vaccine | SARS-CoV-2 virus |
| | | vaccine. | | vaccine. |
| 5 | recombinant human | recombinant human | recombinant human | recombinant human |
| | adenovirus (hAd- | adenovirus (hAd- | adenovirus (hAd- | adenovirus (hAd- |
| | SARS-CoV-2-S) | SARS-CoV-2-S) | SARS-CoV-2-S) | SARS-CoV-2-S) |
| 6 | recombinant | recombinant | recombinant | recombinant |
| | chimpanzee | chimpanzee | chimpanzee | chimpanzee |
| | adenovirus (ChAd- | adenovirus (ChAd- | adenovirus (ChAd- | adenovirus (ChAd- |
| | SARS-CoV-2-S) | SARS-CoV-2-S) | SARS-CoV-2-S) | SARS-CoV-2-S) |
| 7 | recombinant human | recombinant | recombinant human | recombinant |
| | adenovirus (hAd- | chimpanzee | adenovirus (hAd- | chimpanzee |
| | SARS-CoV-2-S) | adenovirus (ChAd- | SARS-CoV-2-S) | adenovirus (ChAd- |
| | | SARS-CoV-2-S) | | SARS-CoV-2-S) |
| 8 | recombinant human | recombinant human | recombinant | recombinant |
| | adenovirus (hAd- | adenovirus (hAd- | chimpanzee | chimpanzee |
| | SARS-CoV-2-S) | SARS-CoV-2-S) | adenovirus (ChAd- | adenovirus (ChAd- |
| | | | SARS-CoV-2-S) | SARS-CoV-2-S) |
| 9 | recombinant | recombinant | recombinant human | recombinant human |
| | chimpanzee | chimpanzee | adenovirus (hAd- | adenovirus (hAd- |
| | adenovirus (ChAd- | adenovirus (ChAd- | SARS-CoV-2-S) | SARS-CoV-2-S) |
| | SARS-CoV-2-S) | SARS-CoV-2-S) | | |
| 10 | killed-inactivated | killed-inactivated | recombinant human | recombinant human |
| | SARS-CoV-2 whole- | SARS-CoV-2 whole- | adenovirus (hAd- | adenovirus (hAd- |
| | virion vaccine | virion vaccine | SARS-CoV-2-S) | SARS-CoV-2-S) |
| 11 | killed-inactivated | killed-inactivated | recombinant | recombinant |
| | SARS-CoV-2 whole- | SARS-CoV-2 whole- | chimpanzee | chimpanzee |
| | virion vaccine | virion vaccine | adenovirus (ChAd- | adenovirus (ChAd- |
| | | | SARS-CoV-2-S) | SARS-CoV-2-S) |
| 12 | recombinant human | recombinant human | killed-inactivated | recombinant |
| | adenovirus (hAd- | adenovirus (hAd- | SARS-CoV-2 whole- | chimpanzee |
| | SARS-CoV-2-S) | SARS-CoV-2-S) | virion vaccine | adenovirus (ChAd- |
| | | | | SARS-CoV-2-S) |
| 13 | killed-inactivated | recombinant | killed-inactivated | recombinant |
| | SARS-CoV-2 whole- | chimpanzee | SARS-CoV-2 whole- | chimpanzee |
| | virion vaccine | adenovirus (ChAd- | virion vaccine | adenovirus (ChAd- |
| | | SARS-CoV-2-S) | | SARS-CoV-2-S) |
| 14 | killed-inactivated | killed-inactivated | recombinant | recombinant |
| | SARS-CoV-2 whole- | SARS-CoV-2 whole- | chimpanzee | chimpanzee |
| | virion vaccine | virion vaccine | adenovirus (ChAd- | adenovirus (ChAd- |
| | | | SARS-CoV-2-S) | SARS-CoV-2-S) |
| 15 | killed-inactivated | recombinant human | killed-inactivated | recombinant |
| | SARS-CoV-2 whole- | adenovirus (hAd- | SARS-CoV-2 whole- | chimpanzee |
| | virion vaccine | SARS-CoV-2-S) | virion vaccine | adenovirus (ChAd- |
| | | | | SARS-CoV-2-S) |

The present vaccine induces enhanced cross protection against all known COVID-19 variants including Delta and Omicron.

### BRIEF DESCRIPTION OF FIGURES

FIG. 1: Diagrammatic representation of heterologous vaccination regimen in general.

FIG. 2: Characterization of BBV154 vaccine candidate

FIG. 3: Immunogenicity of single dose vaccination of BBV154

FIG. 4: Generation of BBV153 vaccine

FIG. 5: Immunogenicity of repeated dose vaccination of BBV154

FIG. 6: IN administration of BBV154 did not induce ChAd36 neutralizing antibodies even after repeated doses.

FIG. 7: Diagram of heterologous routes of vaccination of BBV154

FIG. 8: IgG and IgA and Neutralization antibody titers elicited by BBV154 heterologous vaccination.

FIG. 9: Diagrammatic representation of heterologous routes of vaccination of BBV153

FIG. 10: IgG and IgA titers elicited by BBV153 heterologous vaccination.

FIG. 11: Diagram of heterologous vaccination regimen of COVAXIN and BBV154.

FIG. 12: Diagram of heterologous vaccination regimen of COVAXIN (two-dose) and BBV154.

FIG. 13: IgG and IgA and Neutralization antibody titers elicited by COVAXIN and BBV154 heterologous vaccination.

FIG. 14: IgG and IgA and Neutralization antibody titers elicited by COVAXIN/COVAXIN and BBV154 heterologous vaccination.

FIG. 15: Surrogate Virus Neutralization (sVNT) titers by COVAXIN and BBV154 heterologous vaccination.

Herein above, BBV154 refers to recombinant chimpanzee adenovirus (ChAd-SARS-CoV-2-S). BBV153 refers to recombinant human adenovirus (hAd5-SARS-CoV-2-S). COVAXIN is a trademark for applicant's killed-inactivated SARS-CoV-2 whole-virion vaccine.

### DESCRIPTION OF THE INVENTION

The present invention discloses homologous or heterologous vaccination regimen to induce an antigen specific immune response to SARS-CoV-2 infection in mammals. Specifically discloses combination of immunogenic compositions and methods of vaccinations for prevention of SARS-CoV-2 infections as well as infections caused by other coronaviruses such as SARS-CoV and MERS-CoV.

More particularly, present invention shows synergism of immunogenicity via combined parental and mucosal immunization against COVID-19.

The invention discloses the effective vaccination strategies including but not limited to administrating same/different vaccine against COVID-19 through heterologous route or administrating different vaccine against COVID-19 through homologous route of administration.

The present invention aims to prevent transmission or viral replication in both lower and upper respiratory tract and discloses and describes the homologous or heterologous approach to induce mucosal and systemic immune response against SARS CoV-2 infections or COVID-19.

### Immunization of Combination of Vaccine:

The present invention describes methods of vaccinations for prevention of SARS-CoV-2 infections as well as infections caused by other coronaviruses such as SARS-CoV and MERS-CoV. Generally, the method involves administering to the subject an effective amount of two immunogenic compositions through heterologous or homologous routes wherein the first dose is referred as prime dose and further dose is referred as booster dose. Without being limited to, vaccine of primary series may be referred hereinafter as prime dose and vaccine of secondary series may be referred as booster dose. Both prime and booster does can be administered through homologous or heterologous routes. Preferred is heterologous route.

This invention discloses the method for generating a robust, immune response in a vaccinated subject against COVID-19 with a SARS-CoV-2 antigen by administering to human subject at least two doses of similar or heterologous COVID-19 vaccines, wherein the said doses are administered in homologous or heterologous routes.

In one aspect present invention discloses a heterologous vaccines comprising immunogenic compositions for prevention of SARS-CoV-2 infections.

Preferably, present invention discloses an immunogenic system for generating robust immune response in mammals against SARS CoV-2 infection.

Accordingly present invention discloses an immunogenic system for generating robust immune response in mammals against SARS CoV-2 infection through homologous or heterologous administration of primary and secondary series vaccine, wherein the primary and secondary series vaccines comprise:
a. a killed-inactivated SARS-CoV-2 whole-virion vaccine; and
b. a recombinant SARS-CoV-2 virus vaccine,

In the said system, the recombinant adenovectored SARS-CoV-2 virus vaccine may be selected from recombinant chimpanzee adenovirus (ChAd-SARS-CoV-2-S), recombinant human adenovirus (hAd-SARS-CoV-2-S), rabies vectored vaccine, respiratory syncytial virus vectored vaccine (RSV), influenza virus (both A and B strains) and other respiratory virus vectored vaccine containing gene segment for full length or partial spike protein or immunogenic part thereof from SARS-CoV-2 virus.

In one embodiment, the recombinant adenovectored SARS-CoV-2 vaccine is recombinant chimpanzee adenovirus (ChAd-SARS-CoV-2-S) vaccine.

The said recombinant chimpanzee adenovirus (ChAd-SARS-CoV-2-S) vaccine contains genome encoding complete or partial spike protein or immunogenic part thereof from SARS-CoV-2.

In another embodiment the recombinant adenovectored SARS-CoV-2 vaccine is recombinant human adenovirus (hAd-SARS-CoV-2-S).

The recombinant adenovectored SARS-CoV-2 vaccine is recombinant human adenovirus (hAd-SARS-CoV-2-S) containing nucleic acid encoding full length codon optimized spike protein of SARS-CoV-2 (rAd-nCoV-Spike).

In one embodiment, the killed-inactivated SARS-CoV-2 whole-virion vaccine is an injection for intramuscular or intradermal administration and the recombinant adenovectored SARS-CoV-2 virus vaccine is a nasal vaccine for intranasal or oral or mucosal administration.

In another embodiment, the killed-inactivated SARS-CoV-2 whole-virion vaccine is a nasal vaccine for intranasal or oral or mucosal administration and the recombinant adenovectored SARS-CoV-2 virus vaccine is an injection for intramuscular or intradermal administration.

In one embodiment vaccine from primary and secondary series comprises killed-inactivated SARS-CoV-2 whole-virion vaccine.

In another embodiment vaccine from primary and secondary series comprises recombinant adenovectored SARS-CoV-2 virus vaccine.

In one embodiment of the invention, the vaccine from primary and secondary series comprises a recombinant human adenovirus that contains gene segment for full length or partial genome encoding complete or partial spike protein or immunogenic part thereof from SARS-CoV-2 virus [(hAd5-SARS-CoV-2-S) BBV153].

In one embodiment of the invention, the vaccine from primary and secondary series comprises of a recombinant chimpanzee adenovirus that contains gene segment for full length or partial genome encoding complete or partial spike protein or immunogenic part thereof from SARS-CoV-2 virus [(ChAd-SARS-CoV-2-S) BBV154].

In one embodiment of the invention, vaccine from primary and secondary series comprises of killed-inactivated SARS-CoV-2 whole-virion vaccine (BBV152).

In one embodiment of the invention, the vaccine from primary series comprises of killed-inactivated recombinant respiratory syncytial virus (RSV) that contains gene segment of full length or partial spike protein or immunogenic part thereof from SARS-CoV-2 or similar virus and the vaccine from secondary series encompasses live recombinant RSV with or without the genome segment of full length or partial spike protein or immunogenic part thereof from SARS-CoV-2 or similar virus.

In one embodiment of the invention, the vaccine from primary series comprises of killed-inactivated influenza virus (both A and B strains) or other respiratory virus vectored vaccine containing gene segment of full length or partial spike glycoprotein or immunogenic part thereof from SARS-CoV-2 and the vaccine from secondary series comprises of live-attenuated (replication-defective and/or codon-deoptimized) influenza virus (both A and B strains) or other respiratory virus vectored vaccine with or without the genome segment of full length or partial spike glycoprotein or immunogenic part thereof from SARS-CoV-2.

In one embodiment of the invention, the vaccine from primary series comprises of recombinant human adenovirus that contains nucleic acid encoding complete or partial spike protein or immunogenic part thereof from SARS-CoV-2 virus [(hAd5-SARS-CoV-2-S) BBV153] and the vaccine from secondary series encompasses recombinant chimpanzee adenovirus that contains nucleic acid encoding complete or partial spike protein or immunogenic part thereof from SARS-CoV-2 virus [(ChAd-SARS-CoV-2-S) BBV154]; or vice versa.

In one embodiment of the invention, the vaccine from primary series comprises of killed-inactivated SARS-CoV-2 whole-virion vaccine (BBV152) and the vaccine from secondary series encompasses recombinant chimpanzee or human adenovirus or RSV or influenza virus with or without the genome encoding complete or partial spike protein or immunogenic part thereof from SARS-CoV-2 or similar virus.

Further the primary and secondary series vaccines may comprise nucleic acid- (DNA or mRNA) or protein [subunit (spike, RBD, S1)] based COVID-19 vaccines.

In one embodiment, the vaccine from primary series comprises of nucleic acid- (DNA or mRNA) or protein [subunit (spike, RBD, S1)] based COVID-19 vaccines and the vaccine from secondary series encompasses recombinant chimpanzee or human adenovirus or RSV or influenza virus with or without the genome encoding complete or partial spike protein or immunogenic part thereof from SARS-CoV-2 or similar virus.

In one of the preferred embodiments, the vaccine from primary series comprises of killed-inactivated SARS-CoV-2 whole-virion vaccine (BBV152) and the vaccine from secondary series encompasses recombinant chimpanzee adenovirus that contains nucleic acid encoding complete or partial spike protein or immunogenic part thereof from SARS-CoV-2 virus [(ChAd-SARS-CoV-2-S) BBV154]; or vice versa.

In another preferred embodiment the vaccine from primary series comprises of killed-inactivated SARS-CoV-2 whole-virion vaccine (BBV152) and the vaccine from secondary series encompasses recombinant human adenovirus (hAd5-SARS-CoV-2-S) containing nucleic acid encoding full length codon optimized spike protein of SARS-CoV-2 (rAd-nCoV-Spike) (BBV153); or vice versa.

According to the preferred embodiment, the primary and secondary series vaccines may be selected from—

| | Primary series | | Secondary series | |
|---|---|---|---|---|
| 1 | recombinant | recombinant | recombinant | recombinant |
| | adenovectored | adenovectored | adenovectored | adenovectored |
| | SARS-CoV-2 virus | SARS-CoV-2 virus | SARS-CoV-2 virus | SARS-CoV-2 virus |
| | vaccine. | vaccine. | vaccine. | vaccine. |
| 2 | killed-inactivated | killed-inactivated | killed-inactivated | killed-inactivated |
| | SARS-CoV-2 whole- | SARS-CoV-2 whole- | SARS-CoV-2 whole- | SARS-CoV-2 whole- |
| | virion vaccine | virion vaccine | virion vaccine | virion vaccine |
| 3 | killed-inactivated | killed-inactivated | recombinant | recombinant |
| | SARS-CoV-2 whole- | SARS-CoV-2 whole- | adenovectored | adenovectored |
| | virion vaccine | virion vaccine | SARS-CoV-2 virus | SARS-CoV-2 virus |
| | | | vaccine. | vaccine. |
| 4 | killed-inactivated | recombinant | killed-inactivated | recombinant |
| | SARS-CoV-2 whole- | adenovectored | SARS-CoV-2 whole- | adenovectored |
| | virion vaccine | SARS-CoV-2 virus | virion vaccine | SARS-CoV-2 virus |
| | | vaccine. | | vaccine. |
| 5 | recombinant human | recombinant human | recombinant human | recombinant human |
| | adenovirus (hAd- | adenovirus (hAd- | adenovirus (hAd- | adenovirus (hAd- |
| | SARS-CoV-2-S) | SARS-CoV-2-S) | SARS-CoV-2-S) | SARS-CoV-2-S) |
| 6 | recombinant | recombinant | recombinant | recombinant |
| | chimpanzee | chimpanzee | chimpanzee | chimpanzee |
| | adenovirus (ChAd- | adenovirus (ChAd- | adenovirus (ChAd- | adenovirus (ChAd- |
| | SARS-CoV-2-S) | SARS-CoV-2-S) | SARS-CoV-2-S) | SARS-CoV-2-S) |
| 7 | recombinant human | recombinant | recombinant human | recombinant |
| | adenovirus (hAd- | chimpanzee | adenovirus (hAd- | chimpanzee |
| | SARS-CoV-2-S) | adenovirus (ChAd- | SARS-CoV-2-S) | adenovirus (ChAd- |
| | | SARS-CoV-2-S) | | SARS-CoV-2-S) |
| 8 | recombinant human | recombinant human | recombinant | recombinant |
| | adenovirus (hAd- | adenovirus (hAd- | chimpanzee | chimpanzee |
| | SARS-CoV-2-S) | SARS-CoV-2-S) | adenovirus (ChAd- | adenovirus (ChAd- |
| | | | SARS-CoV-2-S) | SARS-CoV-2-S) |
| 9 | recombinant | recombinant | recombinant human | recombinant human |
| | chimpanzee | chimpanzee | adenovirus (hAd- | adenovirus (hAd- |
| | adenovirus (ChAd- | adenovirus (ChAd- | SARS-CoV-2-S) | SARS-CoV-2-S) |
| | SARS-CoV-2-S) | SARS-CoV-2-S) | | |
| 10 | killed-inactivated | killed-inactivated | recombinant human | recombinant human |
| | SARS-CoV-2 whole- | SARS-CoV-2 whole- | adenovirus (hAd- | adenovirus (hAd- |
| | virion vaccine | virion vaccine | SARS-CoV-2-S) | SARS-CoV-2-S) |
| 11 | killed-inactivated | killed-inactivated | recombinant | recombinant |
| | SARS-CoV-2 whole- | SARS-CoV-2 whole- | chimpanzee | chimpanzee |
| | virion vaccine | virion vaccine | adenovirus (ChAd- | adenovirus (ChAd- |
| | | | SARS-CoV-2-S) | SARS-CoV-2-S) |
| 12 | recombinant human | recombinant human | killed-inactivated | recombinant |
| | adenovirus (hAd- | adenovirus (hAd- | SARS-CoV-2 whole- | chimpanzee |
| | SARS-CoV-2-S) | SARS-CoV-2-S) | virion vaccine | adenovirus (ChAd- |
| | | | | SARS-CoV-2-S) |
| 13 | killed-inactivated | recombinant | killed-inactivated | recombinant |
| | SARS-CoV-2 whole- | chimpanzee | SARS-CoV-2 whole- | chimpanzee |
| | virion vaccine | adenovirus (ChAd- | virion vaccine | adenovirus (ChAd- |
| | | SARS-CoV-2-S) | | SARS-CoV-2-S) |
| 14 | killed-inactivated | killed-inactivated | recombinant | recombinant |
| | SARS-CoV-2 whole- | SARS-CoV-2 whole- | chimpanzee | chimpanzee |
| | virion vaccine | virion vaccine | adenovirus (ChAd- | adenovirus (ChAd- |
| | | | SARS-CoV-2-S) | SARS-CoV-2-S) |
| 15 | killed-inactivated | recombinant human | killed-inactivated | recombinant |
| | SARS-CoV-2 whole- | adenovirus (hAd- | SARS-CoV-2 whole- | chimpanzee |
| | virion vaccine | SARS-CoV-2-S) | virion vaccine | adenovirus (ChAd- |
| | | | | SARS-CoV-2-S) |

According to present invention the second vaccine of primary series is administered between 4-10 weeks after the first vaccine of primary series. Further the first vaccine of secondary series is administered no less than about 10 weeks after the second vaccine of primary series.

The dose concentration of killed-inactivated SARS-CoV-2 whole-virion vaccine in the present context is in the range of 5 μg/dose-7 μg/dose in 0.5 mL volume.

In the preferred embodiment, the dose concentration of killed-inactivated SARS-CoV-2 whole-virion vaccine is 6 μg/dose in 0.5 mL volume.

Further the dose concentration of (ChAd-SARS-CoV-2-S) vaccine is in the range of 10¹⁰ VP/dose-10¹² VP/dose in 0.2-0.5 ml dose.

In the preferred embodiment, the dose concentration of (ChAd-SARS-CoV-2-S) vaccine is 1×10¹¹ VP/dose at 0.5 mL volume.

In another embodiment of the invention, the vaccines from primary and secondary series are administered intranasal using nasal drops or nasal spray device.

The vaccines in the said heterologous vaccination are stable at 2-8° C.

In another aspect of the invention, said combination of vaccine when administered in heterologous regimen elicits increased IgG and IgA antibody response compared to homologous regimen.

The heterologous route of administration of vaccines elicits increased neutralizing antibody response compared to homologous regimen.

The heterologous administration of the vaccines elicits increased mucosal T cell response than homologous route.

In one aspect present invention discloses a combination of pharmaceuticals comprising immunogenic compositions formulated with or without adjuvant.

In one embodiment the vaccine from primary series is administered through intramuscular route. In another embodiment of the invention, vaccine from primary series is administered through intradermal route. However, the vaccine from secondary series is administered through intranasal route. In another embodiment of the invention, the vaccine from secondary series is administered through oral route.

In another embodiment the vaccine from primary series is administered through intranasal route. In another embodiment of the invention, vaccine from primary series is administered through oral route. However, the vaccine from secondary series is administered through intramuscular route. In another embodiment of the invention, the vaccine from secondary series is administered through intradermal route.

In one embodiment, the present invention induces mucosal immune response along with systemic immune response.

In one embodiment, the present invention discloses and provides approaches and/or methods to prevent transmission or viral replication in both lower and upper respiratory tract.

In one embodiment, the present invention discloses and provides assess ability of new adjuvant formulations to induce both humoral and cell mediated T cell responses and mucosal responses (IgA titers).

The present invention provides the broad epitope protection from an inactivated IM vaccine (COVAXIN) and the heightened cell mediated responses/mucosal protection conferred from an adenovectored IN vaccine (BBV154) that augments the efficacy of heterologous vaccination schedules.

The said heterologous vaccination of same or different of vaccines induces enhanced cross protection against COVID-19 variants such as Delta or omicron.

### Method of Administration of Vaccine Doses Homologous Route and Heterologous Route:

Present invention discloses and describes the method of generating a robust immune response in mammals, also suitable for humans against COVID-19 by utilizing homologous or heterologous vaccination regimen.

The invention also discloses a method for administering a vaccine from primary series and a vaccine from secondary series through heterologous or homologous routes.

In one aspect the present invention discloses a method of generating robust immune response in mammals against SARS-CoV-2 antigen by administering two or more doses of same or different COVID-19 vaccines through same or different routes, wherein at least one vaccine is selected from a primary series of vaccines and at least one vaccine is selected from a secondary series of vaccines and wherein vaccines of primary and secondary series are administered through homologous or heterologous routes.

The homologous route of administration comprises administering primary and secondary series of vaccines through same route.

In homologous route, the primary and secondary series of vaccines are administered through the same route selected from intramuscular, intradermal, intranasal, oral and mucosal route.

The heterologous route of administration comprises administering primary and secondary series vaccines through different routes.

In heterologous route, at least two vaccines from primary and secondary series are administered through different routes.

In said heterologous route at least one vaccine from primary or secondary series is administered through intramuscular or intradermal route and at least one vaccine from primary or secondary series is administered through intranasal, oral or mucosal route.

In one embodiment, the vaccine of primary series is an injection for intramuscular or intradermal administration and the vaccine of secondary series is a nasal vaccine for intranasal or oral or mucosal administration.

In another embodiment, the vaccine of primary series is a nasal vaccine for intranasal or oral or mucosal administration and the vaccine of secondary series is an injection for intramuscular or intradermal administration.

In one embodiment, both primary and secondary series of vaccines comprise of killed-inactivated SARS-CoV-2 whole-virion vaccine or both primary and secondary series of vaccines comprise of recombinant adenovectored SARS-CoV-2 virus vaccine.

In another embodiment the primary series of vaccine comprises at least one killed-inactivated SARS-CoV-2 whole-virion vaccine and secondary series of vaccine comprises at least one recombinant adenovectored SARS-CoV-2 virus vaccine, or other way around.

In the said method the recombinant adenovectored SARS-CoV-2 virus vaccine comprises of recombinant chimpanzee adenovirus (ChAd-SARS-CoV-2-S), recombinant human adenovirus (hAd-SARS-CoV-2-S), rabies vectored vaccine, respiratory syncytial virus vectored vaccine (RSV), influenza virus (both A and B strains) or other respiratory virus vectored vaccine containing gene segment for full length or partial spike protein or immunogenic part thereof from SARS-CoV-2 virus.

In one embodiment, the recombinant adenovectored SARS-CoV-2 virus vaccine is a recombinant chimpanzee adenovirus containing gene segment for full length or partial genome encoding complete or partial spike protein or immunogenic part thereof from SARS-CoV-2 virus (ChAd-SARS-CoV-2-S).

In another embodiment, the recombinant adenovectored SARS-CoV-2 virus vaccine is a recombinant human adenovirus (hAd-SARS-CoV-2-S) containing nucleic acid encoding full length codon optimized spike protein of SARS-CoV-2 (rAd-nCoV-Spike).

In one embodiment of the invention, both the primary and secondary series of vaccines comprise of recombinant human adenovirus (hAd-SARS-CoV-2-S) with nucleic acid encoding full length codon optimized spike protein of SARS-CoV-2 (rAd-nCoV-Spike) (BBV153).

In one embodiment of the invention, both the primary and secondary series of vaccines comprise of a recombinant chimpanzee adenovirus that contains gene segment for full length or partial genome encoding complete or partial spike protein or immunogenic part thereof from SARS-CoV-2 virus [(ChAd-SARS-CoV-2-S) BBV154].

In one embodiment of the invention, both primary and secondary series of vaccines comprises of killed-inactivated SARS-CoV-2 whole-virion vaccine (BBV152).

In one embodiment of the invention, the vaccine from primary series comprises of killed-inactivated recombinant respiratory syncytial virus (RSV) that contains gene segment of full length or partial spike protein or immunogenic part thereof from SARS-CoV-2 or similar virus and the vaccine from secondary series encompasses live recombinant RSV with or without the genome segment of full length or partial spike protein or immunogenic part thereof from SARS-CoV-2 or similar virus.

In one embodiment of the invention, the vaccine from primary series comprises of killed-inactivated influenza virus (both A and B strains) or other respiratory virus vectored vaccine containing gene segment of full length or partial spike glycoprotein or immunogenic part thereof from SARS-CoV-2 and the vaccine from secondary series comprises of live-attenuated (replication-defective and/or codon-deoptimized) influenza virus (both A and B strains) or other respiratory virus vectored vaccine with or without the genome segment of full length or partial spike glycoprotein or immunogenic part thereof from SARS-CoV-2.

In one embodiment of the invention, the vaccine from primary series comprises of recombinant human adenovirus that contains nucleic acid encoding complete or partial spike protein or immunogenic part thereof from SARS-CoV-2 virus [(hAd5-SARS-CoV-2-S) BBV153] and the vaccine from secondary series encompasses recombinant chimpanzee adenovirus that contains nucleic acid encoding complete or partial spike protein or immunogenic part thereof from SARS-CoV-2 virus [(ChAd-SARS-CoV-2-S) BBV154]; or vice versa.

In one embodiment of the invention, the vaccine from primary series comprises of killed-inactivated SARS-CoV-2 whole-virion vaccine (BBV152) and the vaccine from secondary series encompasses recombinant chimpanzee or human adenovirus or RSV or influenza virus with or without the genome encoding complete or partial spike protein or immunogenic part thereof from SARS-CoV-2 or similar virus.

Further the primary and secondary series of vaccines may comprise nucleic acid-(DNA or mRNA) or protein [subunit (spike, RBD, S1)] based COVID-19 vaccines.

In one embodiment, the vaccine from primary series comprises of nucleic acid- (DNA or mRNA) or protein [subunit (spike, RBD, S1)] based COVID-19 vaccines and the vaccine from secondary series encompasses recombinant chimpanzee or human adenovirus or RSV or influenza virus with or without the genome encoding complete or partial spike protein or immunogenic part thereof from SARS-CoV-2 or similar virus.

In one of the preferred embodiments, the vaccine from primary series comprises of killed-inactivated SARS-CoV-2 whole-virion vaccine (BBV152) and the vaccine from secondary series encompasses recombinant chimpanzee adenovirus that contains nucleic acid encoding complete or partial spike protein or immunogenic part thereof from SARS-CoV-2 virus [(ChAd-SARS-CoV-2-S) BBV154]; or vice versa.

In another preferred embodiment the vaccine from primary series comprises of killed-inactivated SARS-CoV-2 whole-virion vaccine (BBV152) and the vaccine from secondary series encompasses recombinant human adenovirus (hAd5-SARS-CoV-2-S) containing nucleic acid encoding full length codon optimized spike protein of SARS-CoV-2 (rAd-nCoV-Spike) (BBV153); or vice versa.

In one embodiment the vaccine from primary series comprises of killed-inactivated SARS-CoV-2 whole-virion vaccine (BBV152) administered through intramuscular or intradermal route and the vaccine from secondary series encompasses recombinant chimpanzee adenovirus that contains nucleic acid encoding complete or partial spike protein or immunogenic part thereof from SARS-CoV-2 virus [(ChAd-SARS-CoV-2-S) BBV154] administered through intranasal or oral or mucosal route.

In one embodiment the vaccine from primary series comprises recombinant chimpanzee adenovirus that contains nucleic acid encoding complete or partial spike protein or immunogenic part thereof from SARS-CoV-2 virus [(ChAd-SARS-CoV-2-S) BBV154] administered through intranasal or oral or mucosal route, and the vaccine from secondary series encompasses killed-inactivated SARS-CoV-2 whole-virion vaccine (BBV152) administered through intramuscular or intradermal route.

In one embodiment the vaccine from primary series comprises recombinant chimpanzee adenovirus that contains nucleic acid encoding complete or partial spike protein or immunogenic part thereof from SARS-CoV-2 virus [(ChAd-SARS-CoV-2-S) BBV154] administered through intramuscular or intradermal route, and the vaccine from secondary series encompasses killed-inactivated SARS-CoV-2 whole-virion vaccine (BBV152) administered through intranasal or oral or mucosal route.

In one of the preferred embodiments, the primary and secondary series vaccines may be selected from—

| | Primary series | | Secondary series | |
|---|---|---|---|---|
| 1 | recombinant | recombinant | recombinant | recombinant |
| | adenovectored | adenovectored | adenovectored | adenovectored |
| | SARS-CoV-2 virus | SARS-CoV-2 virus | SARS-CoV-2 virus | SARS-CoV-2 virus |
| | vaccine. | vaccine. | vaccine. | vaccine. |
| 2 | killed-inactivated | killed-inactivated | killed-inactivated | killed-inactivated |
| | SARS-CoV-2 whole- | SARS-CoV-2 whole- | SARS-CoV-2 whole- | SARS-CoV-2 whole- |
| | virion vaccine | virion vaccine | virion vaccine | virion vaccine |
| 3 | killed-inactivated | killed-inactivated | recombinant | recombinant |
| | SARS-CoV-2 whole- | SARS-CoV-2 whole- | adenovectored | adenovectored |
| | virion vaccine | virion vaccine | SARS-CoV-2 virus | SARS-CoV-2 virus |
| | | | vaccine. | vaccine. |
| 4 | killed-inactivated | recombinant | killed-inactivated | recombinant |
| | SARS-CoV-2 whole- | adenovectored | SARS-CoV-2 whole- | adenovectored |
| | virion vaccine | SARS-CoV-2 virus | virion vaccine | SARS-CoV-2 virus |
| | | vaccine. | | vaccine. |
| 5 | recombinant human | recombinant human | recombinant human | recombinant human |
| | adenovirus (hAd- | adenovirus (hAd- | adenovirus (hAd- | adenovirus (hAd- |
| | SARS-CoV-2-S) | SARS-CoV-2-S) | SARS-CoV-2-S) | SARS-CoV-2-S) |
| 6 | recombinant | recombinant | recombinant | recombinant |
| | chimpanzee | chimpanzee | chimpanzee | chimpanzee |
| | adenovirus (ChAd- | adenovirus (ChAd- | adenovirus (ChAd- | adenovirus (ChAd- |
| | SARS-CoV-2-S) | SARS-CoV-2-S) | SARS-CoV-2-S) | SARS-CoV-2-S) |
| 7 | recombinant human | recombinant | recombinant human | recombinant |
| | adenovirus (hAd- | chimpanzee | adenovirus (hAd- | chimpanzee |
| | SARS-CoV-2-S) | adenovirus (ChAd- | SARS-CoV-2-S) | adenovirus (ChAd- |
| | | SARS-CoV-2-S) | | SARS-CoV-2-S) |
| 8 | recombinant human | recombinant human | recombinant | recombinant |
| | adenovirus (hAd- | adenovirus (hAd- | chimpanzee | chimpanzee |
| | SARS-CoV-2-S) | SARS-CoV-2-S) | adenovirus (ChAd- | adenovirus (ChAd- |
| | | | SARS-CoV-2-S) | SARS-CoV-2-S) |
| 9 | recombinant | recombinant | recombinant human | recombinant human |
| | chimpanzee | chimpanzee | adenovirus (hAd- | adenovirus (hAd- |
| | adenovirus (ChAd- | adenovirus (ChAd- | SARS-CoV-2-S) | SARS-CoV-2-S) |
| | SARS-CoV-2-S) | SARS-CoV-2-S) | | |
| 10 | killed-inactivated | killed-inactivated | recombinant human | recombinant human |
| | SARS-CoV-2 whole- | SARS-CoV-2 whole- | adenovirus (hAd- | adenovirus (hAd- |
| | virion vaccine | virion vaccine | SARS-CoV-2-S) | SARS-CoV-2-S) |
| 11 | killed-inactivated | killed-inactivated | recombinant | recombinant |
| | SARS-CoV-2 whole- | SARS-CoV-2 whole- | chimpanzee | chimpanzee |
| | virion vaccine | virion vaccine | adenovirus (ChAd- | adenovirus (ChAd- |
| | | | SARS-CoV-2-S) | SARS-CoV-2-S) |
| 12 | recombinant human | recombinant human | killed-inactivated | recombinant |
| | adenovirus (hAd- | adenovirus (hAd- | SARS-CoV-2 whole- | chimpanzee |
| | SARS-CoV-2-S) | SARS-CoV-2-S) | virion vaccine | adenovirus (ChAd- |
| | | | | SARS-CoV-2-S) |
| 13 | killed-inactivated | recombinant | killed-inactivated | recombinant |
| | SARS-CoV-2 whole- | chimpanzee | SARS-CoV-2 whole- | chimpanzee |
| | virion vaccine | adenovirus (ChAd- | virion vaccine | adenovirus (ChAd- |
| | | SARS-CoV-2-S) | | SARS-CoV-2-S) |
| 14 | killed-inactivated | killed-inactivated | recombinant | recombinant |
| | SARS-CoV-2 whole- | SARS-CoV-2 whole- | chimpanzee | chimpanzee |
| | virion vaccine | virion vaccine | adenovirus (ChAd- | adenovirus (ChAd- |
| | | | SARS-CoV-2-S) | SARS-CoV-2-S) |
| 15 | killed-inactivated | recombinant human | killed-inactivated | recombinant |
| | SARS-CoV-2 whole- | adenovirus (hAd- | SARS-CoV-2 whole- | chimpanzee |
| | virion vaccine | SARS-CoV-2-S) | virion vaccine | adenovirus (ChAd- |
| | | | | SARS-CoV-2-S) |

According to present method at least one vaccine is selected from primary series and at least one vaccine is selected from secondary series.

One embodiment of the invention discloses a method of treatment and/or prophylaxis for COVID-19 and/or eliciting an immune response against SARS-CoV-2 and its variants in mammals, using adenovirus vector(s) expression such as Chimpanzee adenovirus (ChAd) engineered to express SARS-CoV-2 spike protein or part/fragment thereof and killed inactivated SARS-CoV-2 whole virion vaccine selected from primary series and secondary series.

One more embodiment of the invention discloses the vaccine from primary series comprising of killed-inactivated SARS-CoV-2 whole-virion vaccine (BBV152) and the vaccine from secondary series comprising of recombinant chimpanzee [(ChAd-SARS-CoV-2-S) BBV154] with the genome encoding complete or partial spike protein or immunogenic part thereof from SARS-CoV-2, or vice versa.

In another embodiment of the invention method is disclosed for eliciting a protective immune response in mammals by administering a vaccine from primary series through parenteral (intramuscular or intradermal route), whereas the vaccine from secondary series is administered via intranasal or oral or mucosal routes or the other way around.

According to the above embodiments, the primary and secondary series vaccines are administered through heterologous routes.

In another embodiment primary and secondary series vaccines are administered through homologous route.

In another embodiment two vaccines of primary series and one vaccine of secondary series is administered through heterologous route.

In one more embodiment one vaccine of primary series and two vaccines are secondary series are administered through heterologous routes.

Further according to present method, the second vaccine of primary series is administered between 4-10 weeks after the first vaccine of primary series.

The first vaccine of secondary series is administered no less than about 10 weeks after the second vaccine of primary series.

In above mentioned embodiments, the dose concentration of BBV154 (ChAd-SARS-CoV-2-S) may range from 10¹⁰ VP/dose-10¹² VP/dose in 0.2-0.5 ml dose, preferably 1×10¹¹ VP/dose at 0.5 mL volume. Further the dose concentration of killed-inactivated SARS-CoV-2 whole-virion vaccine is in the range of 5 μg/dose-7 μg/dose in 0.5 mL volume. Preferably the dose concentration of killed-inactivated SARS-CoV-2 whole-virion vaccine (BBV152) is (6 ug/dose) in 0.5 mL volume.

Further the vaccines selected from primary and secondary series further comprise with or without adjuvant.

In another aspect present invention discloses a method of generating robust immune response in mammals against SARS-CoV-2 antigen by administering one or more killed-inactivated SARS-CoV-2 whole-virion vaccine through intramuscular or intradermal route and one or more recombinant chimpanzee adenovirus (ChAd-SARS-CoV-2-S) vaccine through intranasal or oral or mucosal route.

Herein the recombinant chimpanzee adenovirus contains gene segment encoding complete or partial spike protein or immunogenic part thereof from SARS-CoV-2 virus (ChAd-SARS-CoV-2-S).

The first vaccine of secondary series is administered between 4-10 weeks after the last vaccine of primary series.

In another aspect present invention discloses a method of generating robust immune response in mammals against SARS-CoV-2 antigen by administering one or more recombinant chimpanzee adenovirus (ChAd-SARS-CoV-2-S) vaccine through intranasal or oral or mucosal route and one or more killed-inactivated SARS-CoV-2 whole-virion vaccine through intramuscular or intradermal route.

Herein the recombinant chimpanzee adenovirus contains gene segment encoding complete or partial spike protein or immunogenic part thereof from SARS-CoV-2 virus (ChAd-SARS-CoV-2-S)

The first vaccine of secondary series is administered between 4-10 weeks after the last vaccine of primary series.

In another aspect present invention discloses a method of generating robust immune response in mammals against SARS-CoV-2 antigen by administering one or more recombinant human adenovirus (hAd-SARS-CoV-2-S) vaccine through intramuscular or intradermal route and one or more recombinant human adenovirus (hAd-SARS-CoV-2-S) vaccine through intranasal or oral or mucosal route.

In another aspect present invention discloses a method of generating robust immune response in mammals against SARS-CoV-2 antigen by administering one or more recombinant human adenovirus (hAd-SARS-CoV-2-S) vaccine through intranasal or oral or mucosal route and one or more recombinant human adenovirus (hAd-SARS-CoV-2-S) vaccine through intramuscular or intradermal route.

In another aspect present invention discloses a method of generating robust immune response in mammals against SARS-CoV-2 antigen by administering one or more recombinant human adenovirus (hAd-SARS-CoV-2-S) vaccine through intradermal or intramuscular route and one or more recombinant chimpanzee adenovirus (ChAd-SARS-CoV-2-S) vaccine through intranasal or oral or mucosal route.

In another aspect of the invention, vaccine from primary and secondary series is administered intranasal using nasal drops or nasal spray device.

In another aspect of the invention, said methods, elicits increased IgG and IgA antibody response compared to homologous regimen.

In another aspect of the invention, said methods, elicits neutralizing antibody response compared to homologous regimen.

In another aspect of the invention, said methods, elicits mucosal T cell response compared to homologous regimen.

In another aspect of the invention, said methods, induce superior cross protection against COVID-19 variants such as Delta or omicron

More specifically, invention describes method of inducing robust immune response in mammals, using adenovirus vector(s) expression such as Chimpanzee adenovirus (ChAd) engineered to express SARS-CoV-2 spike protein or part/fragment thereof and SARS-CoV-2 whole virion vaccine. Further invention also disclosed, enhanced cross protection against COVID-19 variants such as Delta and Omicron compared to homologous regime.

In the said method parenteral followed by a mucosal vaccination induces qualitatively and quantitatively better immune response, including significant reduction or prevention of in viral replication in the upper and lower respiratory tracts in mammals, also suitable for immunizing human subjects.

Said method induces mucosal immune response along with systemic immune response.

### Advantages

The main objective in using this approach is to elicit comprehensive and long-lasting immunity.
Combination of antigens and formulations pursues greater levels of immunity compared to the immune response obtained by a single vaccination or by inoculations with the same antigen.
Heterologous (systemic and mucosal) routes of vaccination will elicit local (mucosal) immunity in the respiratory tract along with the longevity of systemic immunity.
Heterologous vaccination overcomes the challenges of shortfall of any particular vaccine.
Use of heterologous vaccines could simplify the logistics issues.
Broad immune response elicited due to heterologous vaccination approach can prevent virus transmission and virus replication.
Use of whole virion inactivated vaccine that comprises multiple antigenic targets may combat SARS CoV-2 Variants of Concern (VOC) in preventing the virus transmission.

### EXAMPLES

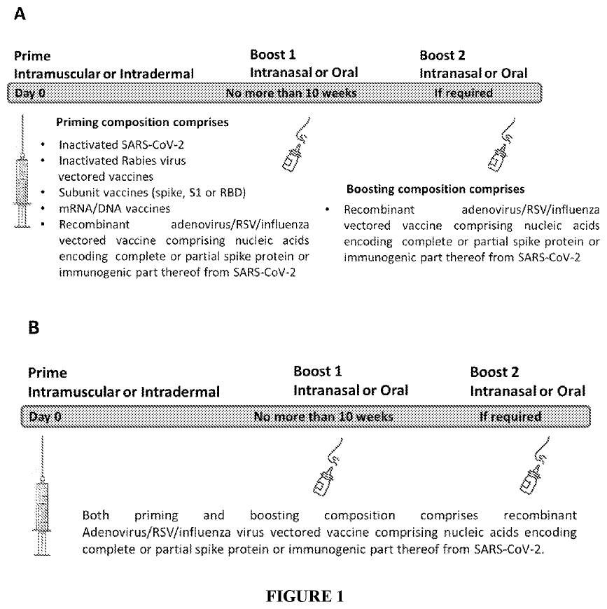

The above describes aspects of the invention further to be understood by the following non-limiting examples 1-5 and corresponding FIGS. 1-15.

### Example 1: Immunogenic System

Present invention discloses an immunogenic system for generating robust immune response in mammals against SARS CoV-2 infection through homologous or heterologous administration of primary and secondary series vaccines.

The said the primary and secondary series vaccines comprise:
a. One or more a killed-inactivated SARS-CoV-2 whole-virion vaccine; and
b. One or more a recombinant adenovectored SARS-CoV-2 virus vaccine.

In the said immunogenic system, the recombinant SARS-CoV-2 virus vaccine may be selected from recombinant chimpanzee adenovirus (ChAd-SARS-CoV-2-S), recombinant human adenovirus (hAd-SARS-CoV-2-S), rabies vectored vaccine, respiratory syncytial virus vectored vaccine (RSV), influenza virus (both A and B strains) and other respiratory virus vectored vaccine containing gene segment for full length or partial spike protein or immunogenic part thereof from SARS-CoV-2 virus.

Preferably, the recombinant SARS-CoV-2 virus vaccine is recombinant chimpanzee adenovirus (ChAd-SARS-CoV-2-S) vaccine that contains genome encoding complete or partial spike protein or immunogenic part thereof from SARS-CoV-2 (BBV154).

Further the recombinant SARS-CoV-2 virus vaccine is recombinant human adenovirus (hAd-SARS-CoV-2-S) containing nucleic acid encoding full length codon optimized spike protein of SARS-CoV-2 (rAd-nCoV-Spike) (BBV153).

Further, for heterologous vaccination study, in said combination, a killed-inactivated SARS-CoV-2 whole-virion vaccine is COVAXIN® (BBV152) (Ella, R. et al. Safety and immunogenicity of an inactivated SARS-CoV-2 vaccine, BBV152: interim results from a double-blind, randomised, multicentre, phase 2 trial, and 3-month follow-up of a double-blind, randomised phase 1 trial, Lancet Infect Dis 21, 950-961, doi:10.1016/S1473-3099(21)00070-0 (2021).) Said vaccine is administered via intramuscular or intradermal route.

According to the present invention, the primary and secondary series vaccines may be selected from—

| | Primary series | | Secondary series | |
|---|---|---|---|---|
| 1 | recombinant | recombinant | recombinant | recombinant |
| | adenovectored | adenovectored | adenovectored | adenovectored |
| | SARS-CoV-2 virus | SARS-CoV-2 virus | SARS-CoV-2 virus | SARS-CoV-2 virus |
| | vaccine. | vaccine. | vaccine. | vaccine. |
| 2 | killed-inactivated | killed-inactivated | killed-inactivated | killed-inactivated |
| | SARS-CoV-2 whole- | SARS-CoV-2 whole- | SARS-CoV-2 whole- | SARS-CoV-2 whole- |
| | virion vaccine | virion vaccine | virion vaccine | virion vaccine |
| 3 | killed-inactivated | killed-inactivated | recombinant | recombinant |
| | SARS-CoV-2 whole- | SARS-CoV-2 whole- | adenovectored | adenovectored |
| | virion vaccine | virion vaccine | SARS-CoV-2 virus | SARS-CoV-2 virus |
| | | | vaccine. | vaccine. |
| 4 | killed-inactivated | recombinant | killed-inactivated | recombinant |
| | SARS-CoV-2 whole- | adenovectored | SARS-CoV-2 whole- | adenovectored |
| | virion vaccine | SARS-CoV-2 virus | virion vaccine | SARS-CoV-2 virus |
| | | vaccine. | | vaccine. |
| 5 | recombinant human | recombinant human | recombinant human | recombinant human |
| | adenovirus (hAd- | adenovirus (hAd- | adenovirus (hAd- | adenovirus (hAd- |
| | SARS-CoV-2-S) | SARS-CoV-2-S) | SARS-CoV-2-S) | SARS-CoV-2-S) |
| 6 | recombinant | recombinant | recombinant | recombinant |
| | chimpanzee | chimpanzee | chimpanzee | chimpanzee |
| | adenovirus (ChAd- | adenovirus (ChAd- | adenovirus (ChAd- | adenovirus (ChAd- |
| | SARS-CoV-2-S) | SARS-CoV-2-S) | SARS-CoV-2-S) | SARS-CoV-2-S) |
| 7 | recombinant human | recombinant | recombinant human | recombinant |
| | adenovirus (hAd- | chimpanzee | adenovirus (hAd- | chimpanzee |
| | SARS-CoV-2-S) | adenovirus (ChAd- | SARS-CoV-2-S) | adenovirus (ChAd- |
| | | SARS-CoV-2-S) | | SARS-CoV-2-S) |
| 8 | recombinant human | recombinant human | recombinant | recombinant |
| | adenovirus (hAd- | adenovirus (hAd- | chimpanzee | chimpanzee |
| | SARS-CoV-2-S) | SARS-CoV-2-S) | adenovirus (ChAd- | adenovirus (ChAd- |
| | | | SARS-CoV-2-S) | SARS-CoV-2-S) |
| 9 | recombinant | recombinant | recombinant human | recombinant human |
| | chimpanzee | chimpanzee | adenovirus (hAd- | adenovirus (hAd- |
| | adenovirus (ChAd- | adenovirus (ChAd- | SARS-CoV-2-S) | SARS-CoV-2-S) |
| | SARS-CoV-2-S) | SARS-CoV-2-S) | | |
| 10 | killed-inactivated | killed-inactivated | recombinant human | recombinant human |
| | SARS-CoV-2 whole- | SARS-CoV-2 whole- | adenovirus (hAd- | adenovirus (hAd- |
| | virion vaccine | virion vaccine | SARS-CoV-2-S) | SARS-CoV-2-S) |
| 11 | killed-inactivated | killed-inactivated | recombinant | recombinant |
| | SARS-CoV-2 whole- | SARS-CoV-2 whole- | chimpanzee | chimpanzee |
| | virion vaccine | virion vaccine | adenovirus (ChAd- | adenovirus (ChAd- |
| | | | SARS-CoV-2-S) | SARS-CoV-2-S) |
| 12 | recombinant human | recombinant human | killed-inactivated | recombinant |
| | adenovirus (hAd- | adenovirus (hAd- | SARS-CoV-2 whole- | chimpanzee |
| | SARS-CoV-2-S) | SARS-CoV-2-S) | virion vaccine | adenovirus (ChAd- |
| | | | | SARS-CoV-2-S) |
| 13 | killed-inactivated | recombinant | killed-inactivated | recombinant |
| | SARS-CoV-2 whole- | chimpanzee | SARS-CoV-2 whole- | chimpanzee |
| | virion vaccine | adenovirus (ChAd- | virion vaccine | adenovirus (ChAd- |
| | | SARS-CoV-2-S) | | SARS-CoV-2-S) |
| 14 | killed-inactivated | killed-inactivated | recombinant | recombinant |
| | SARS-CoV-2 whole- | SARS-CoV-2 whole- | chimpanzee | chimpanzee |
| | virion vaccine | virion vaccine | adenovirus (ChAd- | adenovirus (ChAd- |
| | | | SARS-CoV-2-S) | SARS-CoV-2-S) |
| 15 | killed-inactivated | recombinant human | killed-inactivated | recombinant |
| | SARS-CoV-2 whole- | adenovirus (hAd- | SARS-CoV-2 whole- | chimpanzee |
| | virion vaccine | SARS-CoV-2-S) | virion vaccine | adenovirus (ChAd- |
| | | | | SARS-CoV-2-S) |

Homologous route may be selected from:

| Primary Series | Secondary Series |
|---|---|
| IM/IM | IM |
| IN/IN | IN |

Heterologous route may be selected from:

| Primary Series | Secondary Series |
|---|---|
| IM/IN | |
| IN/IM | |
| IM/IM | IN |
| IM/IM | IM/IN |
| IN/IN | IM |
| IM/IN | IN |
| IN/IM | IN |

IM = Intramuscular or intradermal
IN = Intranasal or mucosal or oral

FIG. 1 shows diagrammatic representation of heterologous vaccination regimen in general. Herein the method involves administering to the subject an effective amount of two or more immunogenic compositions through heterologous or homologous routes wherein the first dose is referred as prime dose and further dose is referred as booster dose.

In the said figure, priming composition comprises of killed-inactivated SARS-CoV-2 whole virion or killed-inactivated rabies virus or recombinant replication defective human/chimpanzee adenovirus or RSV or influenza virus or subunit vaccines or nucleic acid- (DNA or mRNA) that comprises nucleic acid encoding SARS-CoV-2 spike protein, or complete or partial spike protein based COVID-19 vaccines administered intramuscular or intradermal on day 0. One dose of boosting composition comprising recombinant human/chimpanzee adenovirus or RSV or Influenza virus that comprises nucleic acid encoding full length or partial spike protein of SARS-CoV-2 administered intranasal or oral no more than about 10 weeks. Different vaccines via heterologous route of administration (shown in FIG. 1A), same vaccines via different route of administration (shown in FIG. 1B).

The broad epitope protection from an inactivated vaccine (COVAXIN-BBV152) and the heightened cell mediated responses/mucosal protection conferred from an adeno-vectored IN vaccine (BBV154/BBV153) augments the efficacy of heterologous vaccination schedules.

### Example 2: ChAd36-SARS-CoV-2-S (BBV154) Vaccine

ChAd36-SARS-CoV-2-S (BBV154), a replication-defective chimpanzee adenovirus (ChAd)-vectored intranasal (IN) COVID-19 vaccine candidate, encodes a prefusion-stabilized version of the SARS-CoV-2 spike protein containing two proline substitutions in the S2 subunit.

### Manufacture of BBV154 Vaccine Candidate:

The chimpanzee adenovirus 36 vector encoding SARS-CoV-2 pre-fusion stabilized spike protein (ChAd36-SARS-CoV-2-S) and the chimpanzee adenovirus 36 vector control (ChAd36-Control) were obtained from Curiel and Diamond's Labs, Washington University in St Louis, USA.

The adenovirus reference material; wild-type human Adenovirus type 5 (Ad5) (Catalogue No: VR1516) was procured from ATCC, USA.

The HEK cells were obtained from Microbix, Canada and were propagated in DMEM (Gibco, USA), supplemented with 5% heat-inactivated fetal bovine serum (FBS; Gibco), and neomycin (Gibco, USA). The HEK cells were maintained at 37° C. with 5% CO₂. These cells were used as a substrate for the production of the BBV154 and in QC testing.

The HEK cell master and working cell banks were prepared in a GMP facility. Working cell bank was used for the preparation of the BBV154 master and working virus banks in a GMP facility. The cell banks and the virus banks were stored at the banking facility and characterized as per ICH guidelines for identity, safety and purity. The GMP production of virus bulk drug substance was initiated by growing HEK working cell bank in T-175 cm² flasks and expanding them in CellSTACK® 1, 10 and 40 stacks (Corning Inc., USA). The HEK cells were propagated up to a cell density of 1400 million cells/CS40 and were infected with working virus bank of BBV154 at 1 MOI. Infected cells along with the spent media were harvested when >80% cytopathic effect was reached. The adenovirus particles were released by treatment with lysis buffer for one hour at room temperature. The lysate was clarified by depth filtration to remove cell debris. The clarified lysate was then concentrated using ultrafiltration membranes and buffer exchanged with phosphate buffered saline (pH 7.4). Retentate sample was passed through a size-exclusion matrix to remove impurities. Flow-through from the size-exclusion chromatography was further concentrated, buffer exchanged and passed through a 0.2 micron-filter to obtain the sterile drug substance. The BBV154 drug product was produced by diluting the drug substance to required concentration of total virus particle with formulation buffer and distributed in vials.

### Characterization of BBV154 Vaccine Candidate:

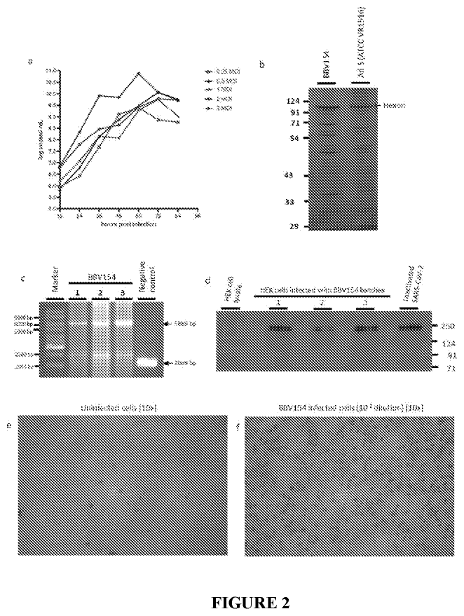

Growth kinetics of BBV154 in HEK cells was studied by infecting HEK cells with 0.25, 0.5, 1, 2 and 3 MOI of BBV154 virus (FIG. 2).

Samples were collected at 12 hours intervals and were subjected to qPCR analysis to estimate the genome copies of the virus. Based on the results of ChAd genome copies (FIG. 2A) and infectious titer (data not shown) estimations of the growth kinetics sample, an optimum MOI of 1 and a harvest time between 60-72 hours were selected.

The downstream purification cascade was followed to obtain a purified and formulated BBV154 vaccine candidate (FIG. 2B). SDS-PAGE and silver staining of BBV154 (drug product) and Ad5 reference reagent from ATCC.

DNA was extracted from three batches of BBV154 and primers flanking the expression cassette were used to detect its presence. Amplification of 5889 bp PCR product indicates the presence of the spike expression cassette. The DNA extracted from ChAd vector was used as negative control which amplified a 2069 bp PCR product which indicates the absence of the spike expression cassette. Marker: Gene ruler SM03111, Thermoscientific (FIG. 2C).

The spike expression by BBV154 was accessed by infection of HEK cells and the cell lysates were subjected to western blotting with antibody against the receptor binding domain (RBD) of spike protein (FIG. 2D). The HEK cells were infected with 5 MOI of three different batches of BBV154 or were left uninfected. Twenty-four hours post-infection the cells were harvested and lysed. The samples were subjected to western blotting with rabbit polyclonal antibody against RBD and anti-rabbit peroxidase conjugated antibody. Inactivated and purified SARS-CoV2 was used as positive control and HEK cell lysate was used as negative control (FIG. 2D). The RBD antibody detected the full-length spike in the cell lysates derived from BBV154 infected cells (FIG. 2D, lanes 2-4) and the spike protein size corresponds to that of the positive control (FIG. 2D, lane 5). The cells were either left uninfected (FIG. 2E) or infected with 10⁻² dilution of BBV154 (FIG. 2F). 48 hours post infection, the cells were fixed and probed with rabbit polyclonal S1 antibody followed by anti-rabbit IgG peroxidase. The peroxidase activity was detected by 3-Amino-9-Ethylcarbazole. The spike expression of BBV154 was also demonstrated by immunocytochemistry, spike expression products being visualized as distinct spots (FIGS. 2E and 2F).

In order to test for the presence of replication competent adenovirus (RCA), BBV154 was passaged in A549 cells that do not have complement E1. As expected, CPE was not observed even after three consecutive passages indicating absence of RCA. To exclude the possibility of inhibition of the RCA in the BBV154 sample, BBV154 spiked with wild type Ad5 (1 or 10 TCID) displayed CPE with an amplification between 10^{9.35} to 10^{10.0} TCID₅₀ (Table 1).

TABLE 1
Test for detection of RCA in the BBV154 sample

| | Number of | | Virus titers after |
|---|---|---|---|
| | passages in | TCID₅₀ assay | three passages |
| Sample Details | A549 cells | performed on | (TCID₅₀) |
| BBV154 sample | 3 | A549 cells | No CPE |
| alone* | | | |
| BBV154 sample | | | 10¹⁰ |
| spiked with 1 TCID | | | |
| of Ad5 | | | |
| BBV154 sample | | | 10^{10.35} |
| spiked with 10 TCID | | | |
| of Ad5 | | | |
| Ad5 alone 10 TCID | | | 10¹¹ |
| BBV154 sample | | HEK cells | No CPE |
| alone | | | |

*Hexon copies: 10.58 log copies/mL; Spike copies: 10.18 log copies/mL; virus particle by A260: 2.82 × 10¹² virus particle/mL.

### Immunogenicity of Single Dose Vaccination of BBV154

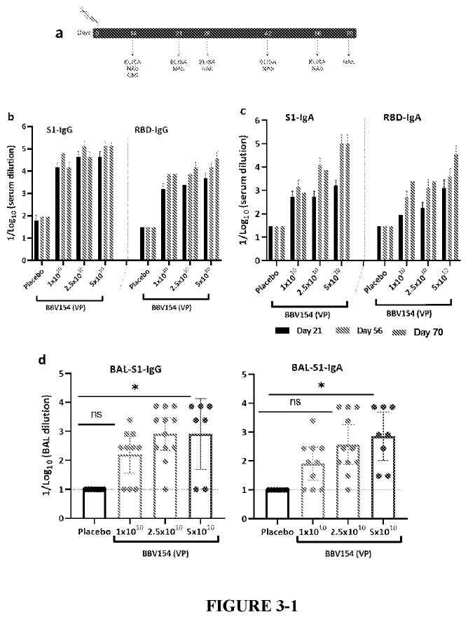

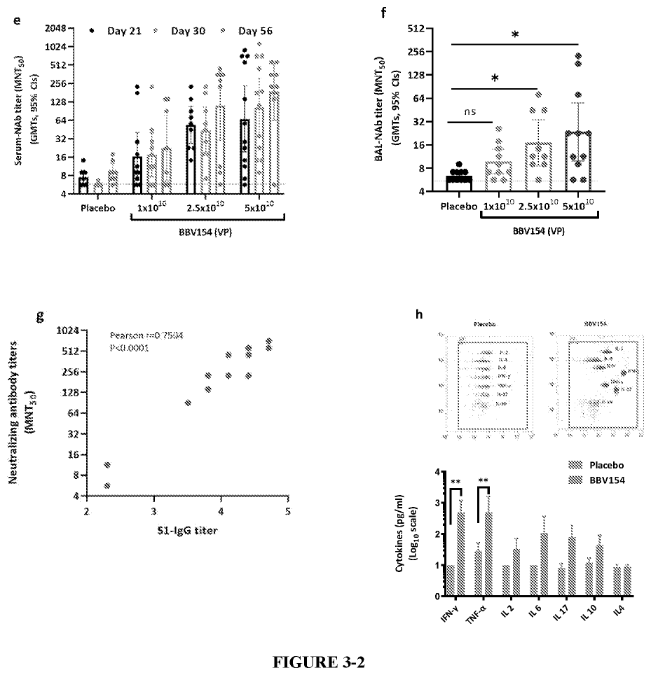

Immunogenicity of BBV154 candidate vaccine was evaluated in BALB/c male and female mice. Schematic diagram of immunization regime mouse experiment is shown in FIG. 3A.

Five to six-week-old BALB/c male and female mice were immunized with 50 μl of placebo or BBV154 via intranasal route either with one-tenth, one-quarter or half of a Human Single Dose (HSD).

Humoral and cellular immune responses were assessed in mice two-weeks post vaccination with either one-tenth, one quarter or half (1×10¹⁰, 2.5×10¹⁰ or 5×10¹⁰ VP respectively) of a human single dose (HSD). Antibody responses in sera of immunized mice at different time points were evaluated (FIGS. 3B & 3C). Mice vaccinated with 5×10¹⁰ VP had higher spike-specific antibody titers than those given 1×10¹⁰ VP, demonstrating a dose-dependent response.

Additionally, substantial increases in serum IgG and IgA levels were observed on day 56 compared with day 21. Spike-specific antibodies were persistent and still measurable on day 70, ten weeks after vaccination, demonstrating durable immunity. IN immunization is known to stimulate mucosal IgA antibodies, providing a first line of defence against respiratory pathogens, and IN vaccination with BBV154 induced S1-specific IgG and IgA responses in the bronchoalveolar lavage (BAL) fluids (FIG. 3E). As with the systemic IgG and IgA, dose-dependent pulmonary antibody responses were observed.

Further, the assessment of SARS-CoV-2 neutralizing antibodies in bronchoalveolar lavage (BAL) fluid of BBV154 immunized mice also displayed SARS-CoV-2 neutralizing antibodies in dose-dependent manner (FIG. 3F).

As expected, neither serum nor BAL fluid from placebo-treated mice exhibited any SARS-CoV-2 neutralizing activity. Further, the level of the neutralizing antibody response was well-correlated with S1-specific systemic IgG levels quantified in individual animals (Pearson r=0.7504, FIG. 3G), signifying that robust antibody responses to spike protein were allied with generation of potentially protective neutralizing antibodies.

Having observed a robust antibody response in vaccinated mice, next examined was the cell mediated immune (CMI) response activated via IN immunization. Ex vivo re-stimulation of splenocytes of vaccinated animals with S1 protein resulted in a significant induction of Th1 associated IFN-γ or TNF-α cytokines (FIG. 3H).

Moreover, T-cells from the BBV154 immune animals produced low levels of IL-10 and IL-4 when compared with T-cells from the placebo-treated mice. This indicates that IN vaccination of BBV154 did not initiate a Th2 response but rather induced the expected antiviral T-cell responses.

### Example 3: Recombinant Human Adenovirus (hAd-SARS-CoV-2-S) Containing Nucleic Acid Encoding Full Length Codon Optimized Spike Protein of SARS-CoV-2 (rAd-nCoV-Spike) (BBV153) Vaccine

### Generation of Replication-Defective Recombinant Adenoviruses Expressing SARS-CoV2 Spike Protein:

Two constructs of recombinant replication-defective hAd vectors based on a human Ad5 virus. One is hAd5-SARS-CoV2-S vector encodes the full-length sequence of SARS-CoV2 S protein and the other is hAd5-SARS-CoV2-S1 vector encodes only S1 region.

Codon optimized SARS-CoV2 Spike sequences were obtained from Genscript and cloned into the shuttle vector (pDC515), which is recombined with the genomic plasmid in 293IQ cells through co-transfection. The plasmids contain frt sites for recombination mediated by the flippase enzyme, which is expressed from the genomic plasmid. Recombination produces a full-length adenovirus with lac operator-controlled expression cassette containing SARS-CoV2 Spike inserted in place of the E1 region. Proteins encoded in the E1 region are essential for replication of adenoviruses, and this function is provided in trans by 293IQ cells, which carry a part of adenoviral genome including the E1 region.

HEK 293 cells were infected with Ad vectors at 5 MOI per cell, and cell lysates were subjected to Western blotting using rabbit anti-Spike serum. Uninfected (HEK 293 lysate) and empty Ad (Ad515) infected cells were used as negative controls. Three different adenoviruses were generated SARS-CoV2 spike [full-length (rAd-nCoV-Spike)]; SARS-CoV-2 S1[(expressing SARS-CoV2 S1 region (rAd-nCoV-S1)] and AdpDC516 (control adenovirus without the transgene).

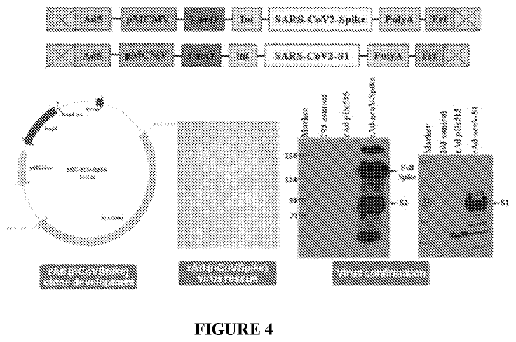

Diagramatic representation of generation of replication-defective recombinant adenoviruses expressing SARS-CoV2 Spike protein (BBV153) is shown in FIG. 4.

### Homologous Administration

Herein the method involves administering to the subject an effective amount of two or more immunogenic compositions through homologous routes wherein the first dose is referred as prime dose and further dose is referred as booster dose.

### Example 4: Immunogenicity of Repeated Dose Vaccination of BBV154

The immunogenicity and tolerability of clinical batch samples of BBV154 vaccine were evaluated in mice, Wistar rats and New Zealand White rabbits with a full human dose (N+1) regimen. Repeated doses of different concentrations of candidate vaccine (5×10⁹ VP [low-dose], 5×10¹⁰ VP [medium-dose], or 5×10¹¹ VP [high-dose] per animal) were administered IN on days 0, 21 and 28.

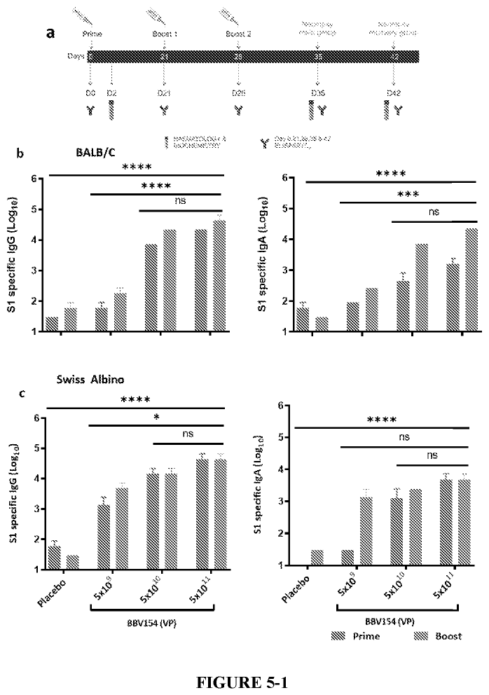

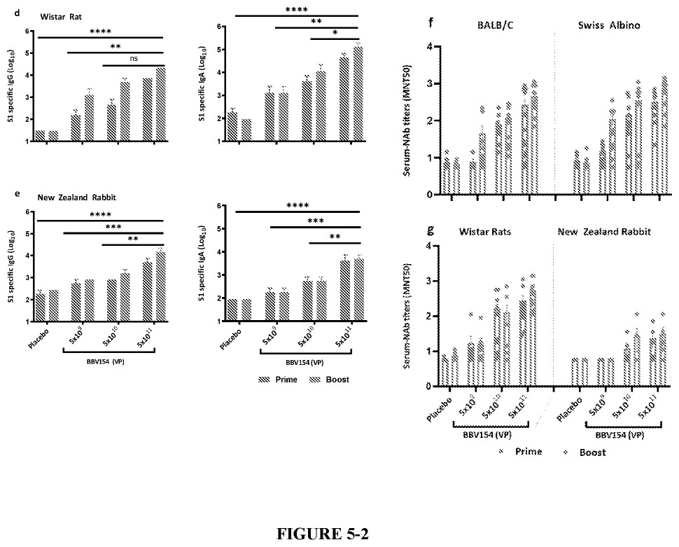

Schematic diagram of repeated dose administration of BBV154 to 6-8-week-old, male and female BALB/c mice or Swiss albino or Wistar rats (n=10-12) or 10-12-week-old male and female New Zealand White rabbits (n=6-8) shown in FIG. 5A. Animals were immunized with placebo or three different concentrations of BBV154 vaccine candidate via the intranasal route in 50 μl (mice), 100 μl (rat), and 200 μl (rabbit) on day 0 and boosted on day 21 (BALB/c mice & Rabbits) or 22 (Swiss albino and rats), and day 28 or 29.

Serum samples were collected 21 days post-primary or pre-prime booster immunization and spike-specific IgG and IgA responses were evaluated by ELISA. Mice, rats and rabbits immunized with high- and medium-doses of vaccine elicited significantly higher IgG and IgA responses against purified S1 antigen than in the low-dose group (FIG. 5B-4F).

Antibody responses in sera of immunized animals at day 21 after priming or at day 28 or 29 after one-week post-boosting were evaluated (FIG. 5B-D). SARS-CoV-2 S1-specific IgG (left panel) and IgA (right panel) levels were measured by ELISA using pooled sera of BALB/c (FIG. 5B), Swiss albino (FIG. 5C), Wistar rats (FIG. 5D) and New Zealand White Rabbits (FIG. 5D). Neutralizing activity of immune serum against SARS-CoV-2 performed by MNT₅₀ at day 21 after priming or at day 28 or 29 after one-week post-boosting (FIG. 5E-F).

Data points represent mean±SEM of individual animal data. Statistical analysis was performed with nonparametric t-test: *, P<0.05; **, P<0.01; ***, P<0.001; ****, P<0.0001.

Consistent with the spike binding antibody response, BBV154 vaccination elicited substantial increases in the SARS-CoV-2 specific neutralizing antibodies in mice, rats and rabbits (FIGS. 5F & 5G). Notably, boosting enhanced serum neutralization activity two- to four-fold in high-dose group (5×10¹¹ VP) animals, with insignificant increases in rabbit immune sera (Table 2), whereas no neutralizing antibodies were detected in sera from placebo-treated animals after primary immunization or boosting.

TABLE 2
SARS-CoV-2 neutralizing antibody responses in
serum following single or double intranasal
administration of candidate vaccine BBV154

| | | Neutralizing Antibody Titers (MNT₅₀) | |
|---|---|---|---|
| | Dose | (mean) | |
| Animal Model | (VP/animal) | Prime | Booster |
| BALB/c | Placebo | 7.6 | 6.7 |
| | 5 × 10⁹ | 7.8 | 45.8 |
| | 5 × 10¹⁰ | 76.6 | 120.9 |
| | 5 × 10¹¹ | 264.8 | 454.1 |
| Swiss Albino | Placebo | 8.6 | 7.2 |
| | 5 × 10⁹ | 13.93 | 111.87 |
| | 5 × 10¹⁰ | 91.25 | 359.5 |
| | 5 × 10¹¹ | 325.15 | 741.11 |
| Wistar Rats | Placebo | 5.66 | 7.44 |
| | 5 × 10⁹ | 17.3 | 16.7 |
| | 5 × 10¹⁰ | 158.7 | 128.25 |
| | 5 × 10¹¹ | 278.3 | 545.2 |
| New Zealand | Placebo | 5.6 | 5.6 |
| Rabbit* | 5 × 10⁹ | 5.6 | 5.6 |
| | 5 × 10¹⁰ | 12.36 | 9.46 |
| | 5 × 10¹¹ | 23.9 | 26.5 |

Each group consisting of 10-12 animals;
*Consisting of 4-6 animals

Following this the levels of anti-vector (ChAd36)-specific neutralizing antibodies in vaccinated animals were assessed. Wistar rats (n=10-12) were immunized IN with BBV154 vaccine as described in FIG. 5A.

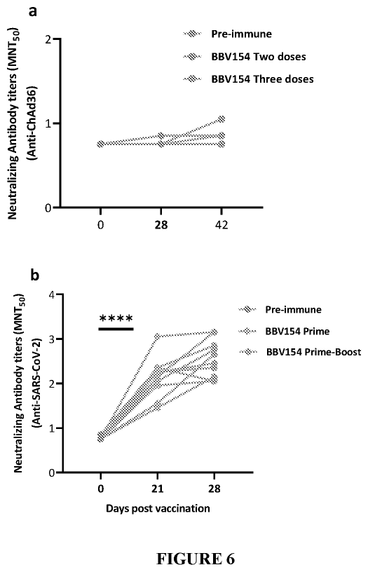

Kinetics of anti-ChAd36 neutralizing antibodies in pre- and post-vaccinated immune sera of rats following three doses of 5×10¹¹ VP of BBV154 is shown in FIG. 6A. Kinetics of anti-SARS-CoV-2 neutralizing antibodies in pre- and post-vaccination sera of rats following two doses of 5×10¹¹ VP of BBV154 is shown in FIG. 6B. Serum samples were assessed using a MNT₅₀ assay. Comparison between different time points were conducted using nonparametric t-test. *, P<0.05; **, P<0.01; ***, P<0.001; ****, P<0.0001.

Most of the animals did not produce the ChAd36 neutralizing antibodies, only 3 out of 12 vaccinated rat sera appearing to have low titers of anti-ChAd36 antibodies (FIG. 6A). However, the immune sera derived from the same animals displayed significantly higher levels of SARS-CoV-2 virus neutralization activity compared with pre-immune sera (FIG. 6B).

Absent or insignificant titers of vector (ChAd36)-specific neutralizing antibodies following three doses of BBV154 implies that IN administration may offer an advantage for repeat vaccination using adenovirus-vectored vaccines.

### Heterologous Administration

Herein the method involves administering to the subject an effective amount of two or more immunogenic compositions through heterologous routes wherein the first dose is referred as prime dose and further dose is referred as booster dose.

### Example 5: Heterologous Route of Administration

### I. Same Vaccine—Heterologous Route of Administration

### ChAd36-SARS-CoV-2-S (BBV154) Vaccine:

### Studies in Mice:

### Diagrammatic Representation of Heterologous Prime-Boost Vaccine Regimen:

Priming composition comprising of recombinant replication defective chimpanzee adenovirus (ChAd36) that comprises nucleic acid encoding full length prefusion spike protein of SARS-CoV-2 [ChAd36-SARS-CoV-2-S (BBV154)] administered IM (Gp-I & III) or IN (Gp-II &IV) on day 0. One dose of boosting of BBV154 administered IM (Gp-I & II) or IN (Gp-III & IV) on day 28, as depicted in FIG. 7.

### Animals and Vaccinations Regimens:

To evaluate the immunogenicity of the heterologous BBV154 prime-boost regimens, four groups of six- to eight-week-old male and female BALB/c mice (Ten mice per group) were primed with 50 ul containing 5×10¹⁰ VP/dose via IM administration (Group I and III) or IN administration (Group II and IV) on day 0. Four weeks later animals were boosted IM (Group-I and II) or IN (Group-III and IV) with BBV154 of same volume and dose. One-week post-booster vaccination SARS-CoV-2 spike specific serum IgG and IgA were assessed by ELISA. SARS-CoV-2 neutralizing antibodies were quantified by MNT, serum-NAb titer and two-weeks post-booster vaccination animals were sacrificed and BAL-NAB titer was analysed.

### Spike-Specific Serum IgG and IgA Quantification:

SARS-CoV-2 spike specific IgG and IgA were measured by ELISA tests as per standard protocols. Briefly, microtiter plates were coated with S1 (Syngene International, India) at a concentration of 2 μg/ml, 80 μL/well in PBS pH 7.4 overnight at 4° C. The plates were then washed three times with 200 μL/well of PBS containing 0.5% SMP and 0.002% tween-20 (Sigma-Aldrich, India). Plates were then blocked at 37° C. for 1 hour with 200 μL/well of PBS containing 3% SMP. Serially diluted pooled or individual sera or BAL samples were added to the plates (100 μL/well) and incubated for 1 hours at 37° C. Secondary antibodies anti-mouse IgG and IgA HRP (Sigma-Aldrich, India) were used. Tetra (3,3′,5,5′) methylbenzidine (Denovo BioLabs Pvt Ltd., India) substrate was added. All samples were read at 450 nm using a microplate reader (iMark, Biorad) following a 5 second mixing. The end-point serum dilution was calculated with three times the standard deviation of the mean optical density (OD) value for pre-immune sera.

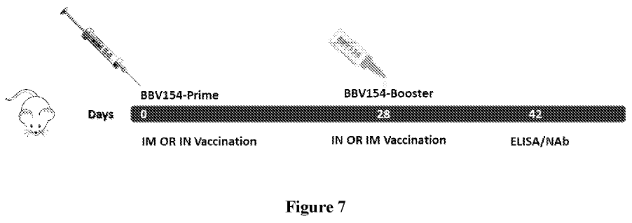

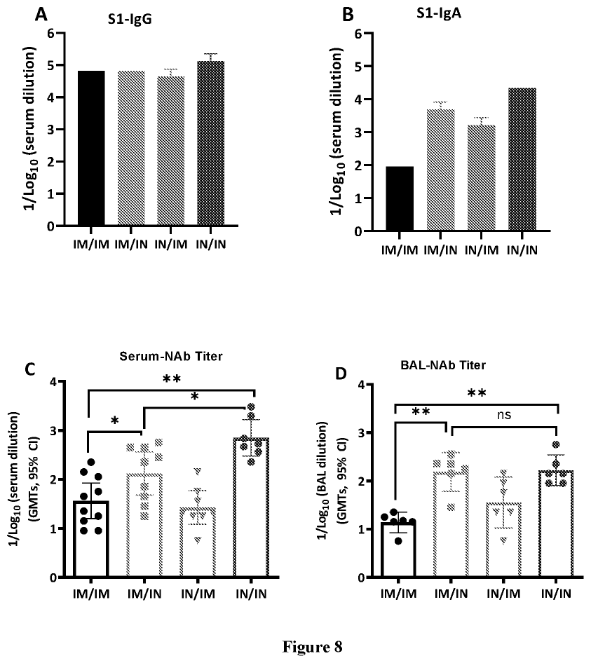

It is observed that heterologous routes of vaccination of BBV154 demonstrated that, compared to IM/IM vaccination, IM-prime-IN-booster regimen induced significantly high levels of S1-specific IgG and IgA (FIGS. 8a and 8b).

### Micro Neutralization Assay (MNT) Using SARS-CoV-2:

The serum and BAL fluids were inactivated in a water bath at 56° C. for 30 min. The heat-inactivated samples were two-fold diluted from 1:8 to 1:4096 and incubated with equal volume of solution containing 100 CCID₅₀ of SARS-CoV-2. After neutralization in a 37° C. incubator for 1 hour, a 1.0×10⁵/mL Vero cell suspension was added to the wells (0.1 mL/well) and cultured in a CO₂ incubator at 37° C. for 3-5 days for SARS-CoV-2. The neutralization end-point was defined as the highest dilution of serum that can protect 50% of the cells from infection by a challenge with 100 CCID₅₀ of SARS-CoV-2. The titers were determined by Karber method. Neutralization antibody potency of <1:20 is negative, while that of >1:20 is positive.

It is observed that IM-prime followed by IN-booster vaccination with BBV154 resulted in significantly high-levels of Nabs in serum as well as BAL (FIGS. 8C and 8D).

In conclusion, compared to IM/IM vaccination, IM-prime-IN-booster regimen induced significantly high levels of S1-specific IgG and IgA and increased neutralizing antibody titers.

rAd-nCoV-Spike [hAd5-SARS-CoV-2-S (BBV153)] Vaccine:

### Diagrammatic Representation of Heterologous Prime-Boost Vaccine Regimen:

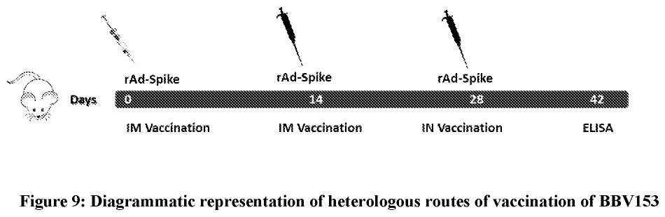

Priming composition comprising of recombinant replication defective human adenovirus (hAd5) that comprises nucleic acid encoding full length codon optimized spike protein of SARS-CoV-2 [rAd-nCoV-Spike (BBV153)] administered IM on days 0 and 14. One dose of boosting of rAd-nCoV-Spike (BBV153) was administered IN on day 28 as depicted in FIG. 9.

### Animals and Vaccinations Regimens:

To evaluate the immunogenicity of the heterologous BBV153 prime-boost regimens, six- to eight-week-old female BALB/c mice (Five mice per group) were primed with 50 ul containing ˜3×10⁸ VP/dose via IM or IN as mentioned in FIG. 9. Two-weeks post-booster 2 vaccination SARS-CoV-2 spike specific serum IgG and IgA were assessed by ELISA.

### Spike-Specific Serum IgG and IgA Quantification:

SARS-CoV-2 spike specific IgG and IgA were measured by ELISA tests as per standard protocols. Briefly, microtiter plates were coated with S1 (Syngene International, India) at a concentration of 2 μg/ml, 80 μL/well in PBS pH 7.4 overnight at 4° C. The plates were then washed three times with 200 μL/well of PBS containing 0.5% SMP and 0.002% tween-20 (Sigma-Aldrich, India). Plates were then blocked at 37° C. for 1 hour with 200 μL/well of PBS containing 3% SMP. Serially diluted pooled or individual sera or BAL samples were added to the plates (100 μL/well) and incubated for 1 hours at 37° C. Secondary antibodies anti-mouse IgG and IgA HRP (Sigma-Aldrich, India) were used. Tetra (3,3′,5,5′) methylbenzidine (Denovo BioLabs Pvt Ltd., India) substrate was added. All samples were read at 450 nm using a microplate reader (iMark, Biorad) following a 5 second mixing. The end-point serum dilution was calculated with three times the standard deviation of the mean optical density (OD) value for pre-immune sera.

### IN Booster Following IM/IM Priming:

Priming composition comprising of recombinant replication defective human adenovirus (hAd) that comprises nucleic acid encoding full length codon optimized spike protein of SARS-CoV-2 [rAd-nCoV-Spike (BBV153)] administered IM on days 0 and 14. One dose of boosting of rAd-nCoV-Spike (BBV153) was administered IN on day 28.

To evaluate the immunogenicity of the rAd-nCoV-Spike vaccine (BBV153) prime-boost regimens, group of six- to eight-week-old female BALB/c mice (five mice per group) were primed with 50 ml containing ˜3×10⁸ VP/dose via IM or IN administered as mentioned above.

Two-weeks post-booster 2 vaccination SARS-CoV-2 spike specific serum IgG and IgA were assessed by ELISA.

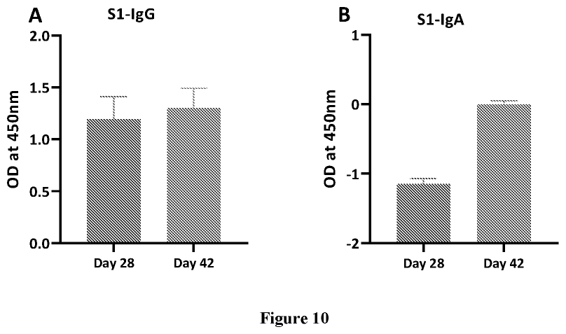

It is observed that IN booster vaccination resulted in spike in IgG (FIG. 10A) and IgA (FIG. 10B) antibodies. IN booster induced significantly high levels of S1-specific IgA.

### II. Different Vaccines-Heterologous Route of Administration Studies in Rabbits

### Diagrammatic Representation of Heterologous Prime—Boost Vaccination Regimen of COVAXIN and BBV154:

Two groups of NZW rabbits (male and female) were immunized IM with COVAXIN, two weeks later animals were administered IN with BBV154. Two-week post-booster vaccination SARS-CoV-2 spike specific IgG and IgA were assessed by ELISA as depicted in FIG. 11.

### Diagrammatic Representation of Heterologous Prime—Boost Vaccination Regimen of COVAXIN (Two-Dose) and BBV154:

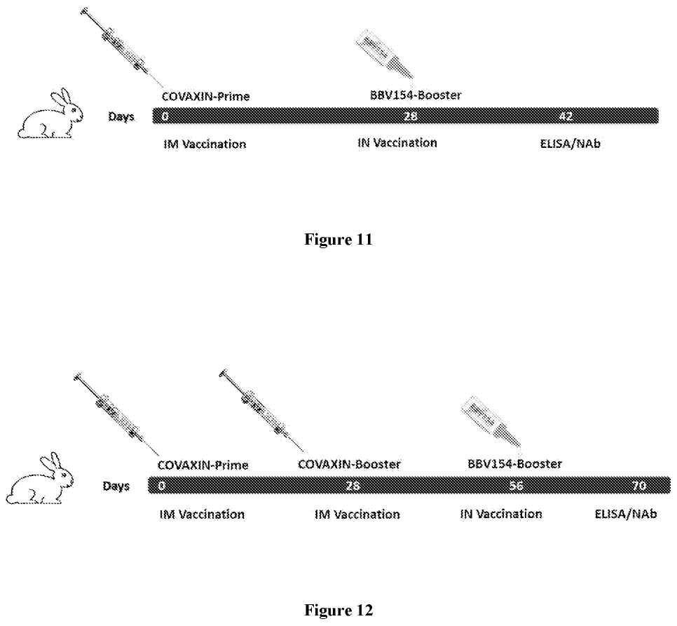

Group of NZW rabbits (male and female) were immunized with two-doses of COVAXIN on days 0 and 28. BBV154 was administered IN on day 42 (two-weeks post booster). Two-week post-booster vaccination (day 70) SARS-CoV-2 spike specific IgG and IgA were assessed by ELISA as depicted in FIG. 12.

### Animals and Vaccinations Regimens:

To assess the immunogenicity of the heterologous COVAXIN/BBV154 prime-boost regimens, 10-12-week-old male and female New Zealand White Rabbits (n=4) in two groups were immunized with heterologous [COVAXIN prime (6 μg/animal) in 0.5 ml and BBV154 booster (1×10¹¹ VP/dose in animal in 0.5 ml] or homologous (COVAXIN prime and COVAXIN booster) regimens on days 0 and 14 or 28.

For two booster dose regime, two groups of NZW rabbits (male and female) were immunized with two-doses of COVAXIN on days 0 and 28. BBV154 was administered IN on day 42 (Two-weeks post booster).

Two-weeks post-booster vaccination SARS-CoV-2 spike specific serum IgG and IgA were assessed by ELISA. SARS-CoV-2 neutralizing antibodies in serum and BAL were quantified by MNT. Percent inhibition of neutralizing antibodies against SARS-CoV-2 delta and omicron variants was performed on day 42.

### Spike-Specific Serum IgG and IgA Quantification:

SARS-CoV-2 spike specific IgG and IgA were measured by ELISA tests as per standard protocols. Briefly, microtiter plates were coated with S1 (Syngene International, India) at a concentration of 2 μg/ml, 80 μL/well in PBS pH 7.4 overnight at 4° C. The plates were then washed three times with 200 μL/well of PBS containing 0.5% SMP and 0.002% tween-20 (Sigma-Aldrich, India). Plates were then blocked at 37° C. for 1 hour with 200 μL/well of PBS containing 3% SMP. Serially diluted pooled or individual sera or BAL samples were added to the plates (100 μL/well) and incubated for 1 hours at 37° C. Secondary antibodies anti-rabbit IgG HRP (Sigma-Aldrich, India) and IgA HRP (Abcam, UK) were used. Tetra (3,3′,5,5′) methylbenzidine (Denovo BioLabs Pvt Ltd., India) substrate was added. All samples were read at 450 nm using a microplate reader (iMark, Biorad) following a 5 second mixing. The end-point serum dilution was calculated with three times the standard deviation of the mean optical density (OD) value for pre-immune sera.

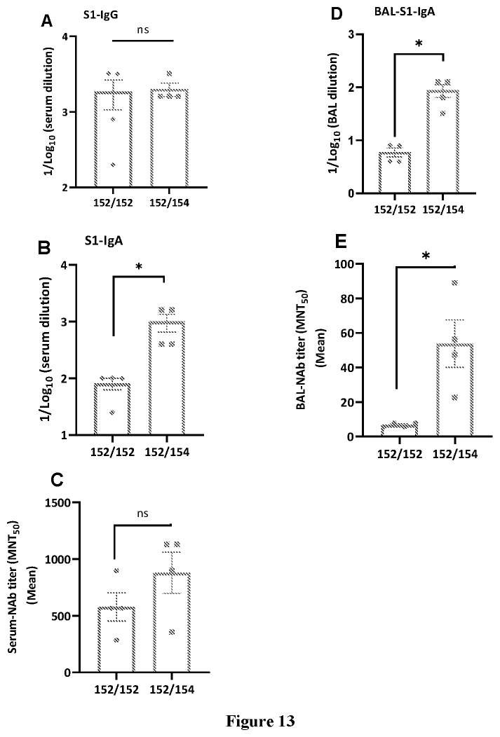

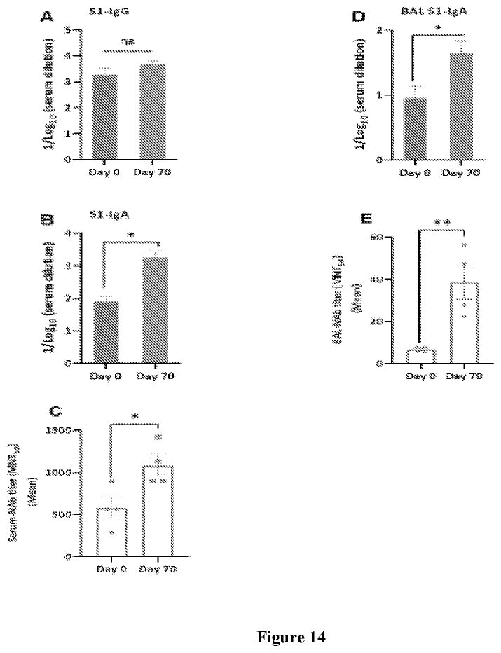

BBV154 IN booster in COVAXIN-primed rabbits elicited comparable levels of spike-specific IgG and remarkably high-levels of serum and BAL IgA titers, as compared with the homologous COVAXIN/COVAXIN immune model (FIGS. 13A, 13B and 13D), whereas BV154 booster following 2 doses of COVAXIN or COVAXIN/COVAXIN priming enhanced the serum and mucosal IgA levels (FIGS. 14A, 14B and 14D).

### Micro Neutralization Assay (MNT) Using SARS-CoV-2:

The serum and BAL fluids were inactivated in a water bath at 56° C. for 30 min. The heat-inactivated samples were two-fold diluted from 1:8 to 1:4096 and incubated with equal volume of solution containing 100 CCID₅₀ of SARS-CoV-2. After neutralization in a 37° C. incubator for 1 hour, a 1.0×10⁵/mL Vero cell suspension was added to the wells (0.1 mL/well) and cultured in a CO₂ incubator at 37° C. for 3-5 days for SARS-CoV-2. The neutralization end-point was defined as the highest dilution of serum that can protect 50% of the cells from infection by a challenge with 100 CCID₅₀ of SARS-CoV-2. The titers were determined by Karber method. Neutralization antibody potency of <1:20 is negative, while that of >1:20 is positive.

BBV154 IN booster in COVAXIN-primed rabbits elicited comparable levels of serum NAb and significant levels of mucosal Nabs in BAL fluid. Whereas BAL fluid from animals immunized with homologous COVAXIN/COVAXIN did not show inhibition of SARS-CoV-2 (FIGS. 13C and 13E), whereas BBV154 booster following 2 doses of COVAXIN or COVAXIN/COVAXIN intranasal booster dose resulted in significant increase in SARS-CoV-2 neutralizing antibodies in serum as well as BAL fluid (FIGS. 14C and 14E).

Surrogate Virus Neutralization (sVNT) Test

Serum samples collected from vaccinated animals were tested for neutralizing activity using the SARS-CoV-2 Surrogate Virus Neutralization Test Kit (cPass Assay, Genscript). Briefly, sera samples were serially diluted (4 fold) with sample dilution buffer (To detect inhibition activity of sera against Omicron, neat & 1:2 diluted sera sample was also tested). Similarly, positive and negative controls provided along with kit were diluted with Sample Dilution Buffer supplied with kit. All diluted sera samples or diluted positive and negative controls were then mixed with 1:1 with HRP conjugated RBD of SARS-CoV-2 D614G or delta (B.1.617.2) or Omicron (B.1.1.529) and incubated at 37° C. for 30 min. Later, 100 μl of each sample/controls were added to the hACE2 coated 96 well plate. Plate was sealed and incubated at 37° C. for 15 min. Wells were then washed 4 times with 260 μl of wash solution. Substrate (TMB) solution (100 μl/well) was added, and the plate was incubated in the dark at room temperature for 15 min. Finally, the reaction was quenched by addition of 50 ul per well of stop solution. Non-linear regression analysis was performed on the transformed data of the absorbance (OD) obtained against D614G and Delta, while normalizing the X-axis. In order to transform the y-axis data, highest absorbance obtained at negative control considered as 0% inhibition and lowest absorbance obtained for positive control considered as 100% inhibition. The reciprocal sera dilution at which 50% inhibition (IC₅₀) was obtained was reported for each sample based on the Non-linear regression analysis.

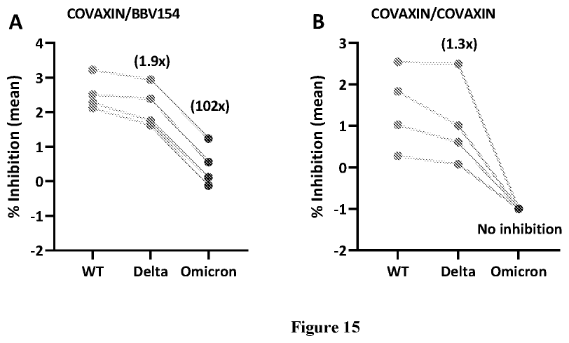

Heterologous boosting with the BBV154 vaccine followed by COVAXIN priming induced a mean titer of NAb of 584.7; 305.5 and 5.75. against B.1, Delta, and omicron, respectively (as shown in FIG. 15).

In Conclusion, Groups of rabbits primed IM with COVAXIN® (Whole-Virion Inactivated vaccine) followed by IN BBV154 booster elicited significantly high levels of spike-specific IgG and IgA titers, compared with the homologous COVAXIN/COVAXIN immune regime. Further, COVAXIN/BBV154 heterologous immune sera showed two-fold higher SARS-CoV-2 neutralizing titers than immune sera derived from rabbits given the COVAXIN/COVAXIN homologous regimen. Whereas BBV154 booster following 2 doses of COVAXIN or COVAXIN/COVAXIN priming enhanced the serum and mucosal IgA levels and resulted in significant increase in SARS-CoV-2 neutralizing antibodies in serum as well as BAL fluid.

Accordingly, BBV154 IN-booster in COVAXIN-primed rabbits elicited superior immune responses, with an acceptable reactogenicity profile comparable to the homologous COVAXIN/COVAXIN vaccination model. Heterologous vaccination of COVAXIN/BBV154 induced neutralizing antibodies against SARS-CoV-2 (delta & omicron) variants with moderate fold-reduction in the NAb titer against delta variant, and higher fold-reduction in the NAb titer against omicron variant as compared with WT. The broad epitope protection from an inactivated vaccine (COVAXIN) and the heightened cell mediated responses/mucosal protection conferred from an adeno-vectored IN vaccine (BBV154) may augment the efficacy of heterologous prime-boost schedules.

Further as the emergence of SARS-CoV-2 variants has raised concerns about the breadth and durability of neutralising antibody responses. To address this, heterologous immune sera was subjected SARS-CoV-2 variants inhibition studies. Heterologous boosting with the BBV154 vaccine followed by COVAXIN priming induced superior protection against SARS CoV-2 variants such as B.1, Delta, and omicron, respectively.

1. A method of generating robust immune response in mammals against SARS-CoV-2 antigen by administering two or more doses of same or different COVID-19 vaccines through same or different routes, wherein at least one vaccine is selected from a primary series of vaccines and at least one vaccine is selected from a secondary series of vaccines and wherein vaccines of primary and secondary series are administered through homologous or heterologous routes.

2. The method as claimed in claim 1, wherein the homologous route of administration comprises administering primary and secondary series of vaccines through same route.

3. The method as claimed in claim 1, wherein the heterologous route of administration comprises administering primary and secondary series vaccines through different routes.

4. The method as claimed in claim 1, wherein in homologous route all vaccines of primary and secondary series are administered through the same route selected from intramuscular, intradermal, intranasal, oral and mucosal route.

5. The method as claimed in claim 1, wherein in heterologous route of administration at least two vaccines from primary and secondary series are administered through different routes.

6. The method as claimed in claim 1, wherein in heterologous route at least one vaccine from primary or secondary series is administered through intramuscular or intradermal route and at least one vaccine from primary or secondary series is administered through intranasal, oral or mucosal route.

7. The method as claimed in claim 1, wherein both primary and secondary series of vaccines comprise of killed-inactivated SARS-CoV-2 whole-virion vaccine.

8. The method as claimed in claim 1, wherein both primary and secondary series of vaccines comprise of recombinant adenovectored SARS-CoV-2 virus vaccine.

9. The method as claimed in claim 1, wherein the primary series of vaccine comprises at least one killed-inactivated SARS-CoV-2 whole-virion vaccine and secondary series of vaccine comprises at least one recombinant adenovectored SARS-CoV-2 virus vaccine.

10. The method as claimed in claim 1, wherein the primary series of vaccine comprises at least one recombinant adenovectored SARS-CoV-2 virus vaccine and secondary series of vaccine comprises at least one killed-inactivated SARS-CoV-2 whole-virion vaccine.

11. The method as claimed in claim 8, wherein recombinant adenovectored SARS-CoV-2 virus vaccine is selected from recombinant chimpanzee adenovirus (ChAd-SARS-CoV-2-S), recombinant human adenovirus (hAd-SARS-CoV-2-S), rabies vectored vaccine, respiratory syncytial virus vectored vaccine (RSV), influenza virus (both A and B strains) or other respiratory virus vectored vaccine containing gene segment for full length or partial spike protein or immunogenic part thereof from SARS-CoV-2 virus.

12. The method as claimed in claim 11, wherein the recombinant adenovectored SARS-CoV-2 virus vaccine is a recombinant chimpanzee adenovirus containing gene segment for full length or partial genome encoding complete or partial spike protein or immunogenic part thereof from SARS-CoV-2 virus (ChAd-SARS-CoV-2-S).

13. The method as claimed in claim 11, wherein the recombinant adenovectored SARS-CoV-2 virus vaccine is a recombinant human adenovirus (hAd-SARS-CoV-2-S) containing nucleic acid encoding full length codon optimized spike protein of SARS-CoV-2 (rAd-nCoV-Spike).

14. The method as claimed in claim 1, wherein both primary and secondary series of vaccines comprise recombinant chimpanzee adenovirus (ChAd-SARS-CoV-2-S) with the genome encoding complete or partial spike protein or immunogenic part thereof from SARS-CoV-2.

15. The method as claimed in claim 1, wherein both primary and secondary series of vaccines comprise of recombinant human adenovirus (hAd-SARS-CoV-2-S) with nucleic acid encoding full length codon optimized spike protein of SARS-CoV-2 (rAd-nCoV-Spike).

16. The method as claimed in claim 1, wherein the primary series of vaccine comprises of killed-inactivated SARS-CoV-2 whole-virion vaccine and the secondary series of vaccine comprises recombinant chimpanzee adenovirus (ChAd-SARS-CoV-2-S) with the genome encoding complete or partial spike protein or immunogenic part thereof from SARS-CoV-2.

17. The method as claimed in claim 1, wherein vaccines of primary and secondary series may be selected from—

| | Primary series | | Secondary series | |
|---|---|---|---|---|
| 1. | recombinant | recombinant | recombinant | recombinant |
| | adenovectored | adenovectored | adenovectored | adenovectored |
| | SARS-CoV-2 virus | SARS-CoV-2 virus | SARS-CoV-2 virus | SARS-CoV-2 virus |
| | vaccine. | vaccine. | vaccine. | vaccine. |
| 2. | killed-inactivated | killed-inactivated | killed-inactivated | killed-inactivated |
| | SARS-CoV-2 whole- | SARS-CoV-2 whole- | SARS-CoV-2 whole- | SARS-CoV-2 whole- |
| | virion vaccine | virion vaccine | virion vaccine | virion vaccine |
| 3. | killed-inactivated | killed-inactivated | recombinant | recombinant |
| | SARS-CoV-2 whole- | SARS-CoV-2 whole- | adenovectored | adenovectored |
| | virion vaccine | virion vaccine | SARS-CoV-2 virus | SARS-CoV-2 virus |
| | | | vaccine. | vaccine. |
| 4. | killed-inactivated | recombinant | killed-inactivated | recombinant |
| | SARS-CoV-2 whole- | adenovectored | SARS-CoV-2 whole- | adenovectored |
| | virion vaccine | SARS-CoV-2 virus | virion vaccine | SARS-CoV-2 virus |
| | | vaccine. | | vaccine. |
| 5. | recombinant human | recombinant human | recombinant human | recombinant human |
| | adenovirus (hAd- | adenovirus (hAd- | adenovirus (hAd- | adenovirus (hAd- |
| | SARS-CoV-2-S) | SARS-CoV-2-S) | SARS-CoV-2-S) | SARS-CoV-2-S) |
| 6. | recombinant | recombinant | recombinant | recombinant |
| | chimpanzee | chimpanzee | chimpanzee | chimpanzee |
| | adenovirus (ChAd- | adenovirus (ChAd- | adenovirus (ChAd- | adenovirus (ChAd- |
| | SARS-CoV-2-S) | SARS-CoV-2-S) | SARS-CoV-2-S) | SARS-CoV-2-S) |
| 7. | recombinant human | recombinant | recombinant human | recombinant |
| | adenovirus (hAd- | chimpanzee | adenovirus (hAd- | chimpanzee |
| | SARS-CoV-2-S) | adenovirus (ChAd- | SARS-CoV-2-S) | adenovirus (ChAd- |
| | | SARS-CoV-2-S) | | SARS-CoV-2-S) |
| 8. | recombinant human | recombinant human | recombinant | recombinant |
| | adenovirus (hAd- | adenovirus (hAd- | chimpanzee | chimpanzee |
| | SARS-CoV-2-S) | SARS-CoV-2-S) | adenovirus (ChAd- | adenovirus (ChAd- |
| | | | SARS-CoV-2-S) | SARS-CoV-2-S) |
| 9. | recombinant | recombinant | recombinant human | recombinant human |
| | chimpanzee | chimpanzee | adenovirus (hAd- | adenovirus (hAd- |
| | adenovirus (ChAd- | adenovirus (ChAd- | SARS-CoV-2-S) | SARS-CoV-2-S) |
| | SARS-CoV-2-S) | SARS-CoV-2-S) | | |
| 10 | killed-inactivated | killed-inactivated | recombinant human | recombinant human |
| | SARS-CoV-2 whole- | SARS-CoV-2 whole- | adenovirus (hAd- | adenovirus (hAd- |
| | virion vaccine | virion vaccine | SARS-CoV-2-S) | SARS-CoV-2-S) |
| 11 | killed-inactivated | killed-inactivated | recombinant | recombinant |
| | SARS-CoV-2 whole- | SARS-CoV-2 whole- | chimpanzee | chimpanzee |
| | virion vaccine | virion vaccine | adenovirus (ChAd- | adenovirus (ChAd- |
| | | | SARS-CoV-2-S) | SARS-CoV-2-S) |
| 12 | recombinant human | recombinant human | killed-inactivated | recombinant |
| | adenovirus (hAd- | adenovirus (hAd- | SARS-CoV-2 whole- | chimpanzee |
| | SARS-CoV-2-S) | SARS-CoV-2-S) | virion vaccine | adenovirus (ChAd- |
| | | | | SARS-CoV-2-S) |
| 13 | killed-inactivated | recombinant | killed-inactivated | recombinant |
| | SARS-CoV-2 whole- | chimpanzee | SARS-CoV-2 whole- | chimpanzee |
| | virion vaccine | adenovirus (ChAd- | virion vaccine | adenovirus (ChAd- |
| | | SARS-CoV-2-S) | | SARS-CoV-2-S) |
| 14 | killed-inactivated | killed-inactivated | recombinant | recombinant |
| | SARS-CoV-2 whole- | SARS-CoV-2 whole- | chimpanzee | chimpanzee |
| | virion vaccine | virion vaccine | adenovirus (ChAd- | adenovirus (ChAd- |
| | | | SARS-CoV-2-S) | SARS-CoV-2-S) |
| 15 | killed-inactivated | recombinant human | killed-inactivated | recombinant |
| | SARS-CoV-2 whole- | adenovirus (hAd- | SARS-CoV-2 whole- | chimpanzee |
| | virion vaccine | SARS-CoV-2-S) | virion vaccine | adenovirus (ChAd- |
| | | | | SARS-CoV-2-S) |

18. The method as claimed in claim 17, wherein at least one vaccine is selected from primary series and at least one vaccine is selected from secondary series.

19. The method as claimed in claim 17, wherein at least one vaccine of primary series is administered through intramuscular or intradermal route and at least one vaccine of secondary series is administered through intranasal or oral or mucosal route.

20. The method as claimed in claim 17, wherein at least one vaccine of primary series is administered through intranasal or oral or mucosal route and at least one vaccine of secondary series is administered through intramuscular or intradermal route.

21. The method as claimed in claim 17, wherein primary and secondary series vaccines are administered through heterologous routes.

22. The method as claimed in claim 17, wherein primary and secondary series vaccines are administered through homologous route.

23. The method as claimed in claim 17, wherein two vaccines of primary series and one vaccine of secondary series is administered through heterologous route.

24. The method as claimed in claim 17, wherein one vaccine of primary series and two vaccines are secondary series are administered through heterologous routes.

25. The method as claimed in claim 1, wherein the second vaccine of primary series is administered between 4-10 weeks after the first vaccine of primary series.

26. The method as claimed in claim 1, wherein the first vaccine of secondary series is administered no less than about 10 weeks after the second vaccine of primary series.

27. The method as claimed in claim 17, wherein the dose concentration of recombinant chimpanzee adenovirus vaccine (ChAd-SARS-CoV-2-S) is between 10¹⁰ VP/dose-10¹² VP/dose in 0.2-0.5 ml dose volume.

28. The method as claimed in claim 27, wherein the dose concentration of (ChAd-SARS-CoV-2-S) vaccine is 1×10¹¹ VP/dose at 0.5 mL volume.

29. The method as claimed in claim 17, wherein the dose concentration of killed-inactivated SARS-CoV-2 whole-virion vaccine is 6 ug/dose in 0.5 mL volume.

30. The method as claimed in claim 17, wherein the primary or secondary series vaccines optionally comprise nucleic acid- (DNA or mRNA) or protein [subunit (spike, RBD, S1)] based COVID-19 vaccines.

31. The method as claimed in claim 1, wherein the method induces superior cross protection against SARS-CoV-2 variants including against Delta and Omicron variants.

32. The method as claimed in claim 1, wherein heterologous route of administration of primary and secondary series vaccines elicits increased IgG and IgA antibody response than homologous route.

33. The method as claimed in claim 1, wherein heterologous route of administration of primary and secondary series vaccine elicits increased neutralizing antibody response compared to homologous regimen.

34. The method as claimed in claim 1, wherein heterologous route of administration of primary and secondary series vaccines elicits increased mucosal T cell response than homologous route.

35. A method of generating robust immune response in mammals against SARS-CoV-2 antigen by administering one or more vaccines of killed-inactivated SARS-CoV-2 whole-virion vaccine through intramuscular or intradermal route and a one or more vaccines of recombinant chimpanzee adenovirus (ChAd-SARS-CoV-2-S) vaccine through intranasal or oral or mucosal route.

36. The method as claimed in claim 35, wherein the recombinant chimpanzee adenovirus contains gene segment encoding complete or partial spike protein or immunogenic part thereof from SARS-CoV-2 virus (ChAd-36-SARS-CoV-2-S)

37. The method as claimed in claims 17 and 35, wherein the first vaccine of secondary series is administered between 4-10 weeks after the last vaccine of primary series.

38. A method of generating robust immune response in mammals against SARS-CoV-2 antigen by administering one or more recombinant chimpanzee adenovirus (ChAd-SARS-CoV-2-S) vaccine through intranasal or oral or mucosal route and one or more killed-inactivated SARS-CoV-2 whole-virion vaccine through intramuscular or intradermal route.

39. The method as claimed in claim 38, wherein the recombinant chimpanzee adenovirus contains gene segment encoding complete or partial spike protein or immunogenic part thereof from SARS-CoV-2 virus (ChAd-SARS-CoV-2-S)

40. The method as claimed in claims 17 and 38, wherein the first vaccine of secondary series is administered between 4-10 weeks after the last vaccine of primary series.

41. A method of generating robust immune response in mammals against SARS-CoV-2 antigen by administering one or more recombinant human adenovirus (hAd-SARS-CoV-2-S) vaccine through intramuscular or intradermal route and one or recombinant human adenovirus (hAd-SARS-CoV-2-S) vaccine through intranasal or oral or mucosal route.

42. A method of generating robust immune response in mammals against SARS-CoV-2 antigen by administering one or more recombinant human adenovirus (hAd-SARS-CoV-2-S) vaccine through intranasal or oral or mucosal route and one or more recombinant human adenovirus (hAd-SARS-CoV-2-S) vaccine through intramuscular or intradermal route.

43. A method of generating robust immune response in mammals against SARS-CoV-2 antigen by administering one or more recombinant human adenovirus (hAd-SARS-CoV-2-S) vaccine through intradermal or intramuscular route and one or more recombinant chimpanzee adenovirus (ChAd-SARS-CoV-2-S) vaccine through intranasal or oral or mucosal route.

44. An immunogenic system for generating robust immune response in mammals against SARS CoV-2 infection through homologous or heterologous administration of primary and secondary series vaccines, wherein the primary and secondary series vaccines comprise:
a. One or more killed-inactivated SARS-CoV-2 whole-virion vaccine; and
b. One or more recombinant adenovectored SARS-CoV-2 virus vaccine;

45. The system as claimed in claim 44, wherein the recombinant adenovectored SARS-CoV-2 virus vaccine may be selected from recombinant chimpanzee adenovirus (ChAd-SARS-CoV-2-S), recombinant human adenovirus (hAd-SARS-CoV-2-S), rabies vectored vaccine, respiratory syncytial virus vectored vaccine (RSV), influenza virus (both A and B strains) and other respiratory virus vectored vaccine containing gene segment for full length or partial spike protein or immunogenic part thereof from SARS-CoV-2 virus.

46. The system as claimed in claim 45, wherein the recombinant adenovectored SARS-CoV-2 vaccine is recombinant chimpanzee adenovirus (ChAd-SARS-CoV-2-S) vaccine.

47. The system as claimed in claim 46, wherein the recombinant chimpanzee adenovirus (ChAd-SARS-CoV-2-S) vaccine contains genome encoding complete or partial spike protein or immunogenic part thereof from SARS-CoV-2.

48. The system as claimed in claim 45, wherein the recombinant adenovectored SARS-CoV-2 vaccine is recombinant human adenovirus (hAd-SARS-CoV-2-S).

49. The system as claimed in claim 48, wherein the recombinant adenovectored SARS-CoV-2 vaccine is recombinant human adenovirus (hAd-SARS-CoV-2-S) containing nucleic acid encoding full length codon optimized spike protein of SARS-CoV-2 (rAd-nCoV-Spike).

50. The system as claimed in claim 44, wherein the dose concentration of killed-inactivated SARS-CoV-2 whole-virion vaccine is in the range of 5 μg/dose-7 μg/dose in 0.5 mL volume.

51. The system as claimed in claim 50, wherein the dose concentration of killed-inactivated SARS-CoV-2 whole-virion vaccine is 6 μg/dose in 0.5 mL volume.

52. The system as claimed in claim 44, wherein the dose concentration of recombinant adenovectored SARS-CoV-2 virus vaccine is in the range of 10¹⁰ VP/dose-10¹² VP/dose in 0.2-0.5 ml dose.

53. The system as claimed in claim 52, wherein the dose concentration of recombinant adenovectored SARS-CoV-2 virus vaccine is 1×10¹¹ VP/dose at 0.5 mL volume.

54. The system as claimed in claim 44, wherein the killed-inactivated SARS-CoV-2 whole-virion vaccine is an injection for intramuscular or intradermal administration.

55. The system as claimed in claim 44, wherein the recombinant adenovectored SARS-CoV-2 vaccine is a nasal vaccine for intranasal or oral or mucosal administration.

56. The system as claimed in claim 44, wherein the killed-inactivated SARS-CoV-2 whole-virion vaccine is a nasal vaccine for intranasal or oral or mucosal administration.

57. The system as claimed in claim 44, wherein the recombinant adenovectored SARS-CoV-2 vaccine is an injection for intramuscular or intradermal administration.

58. The system as claimed in claim 44, wherein the system optionally comprises nucleic acid vaccines such as DNA or mRNA; or protein such as subunit spike, RBD or S1.

59. The system as claimed in claim 44, wherein the vaccines are formulated with an adjuvant.

60. The system as claimed in claim 44, wherein the vaccines are formulated without any adjuvant.

61. The system as claimed in claim 44, wherein the vaccines are stable at 2-8° C.

62. The system as claimed in claim 44, wherein heterologous administration of the vaccines elicits increased IgG and IgA antibody response than homologous route.

63. The system as claimed in claim 44, wherein heterologous administration of the vaccines elicits increased neutralizing antibody response compared to homologous regimen.

64. The system as claimed in claim 44, wherein heterologous administration of the vaccines elicits increased mucosal T cell response than homologous route.

65. The system as claimed in claim 44, wherein the system induces enhanced cross protection against all known COVID-19 variants including Delta and Omicron.

66. The system as claimed in claim 44, wherein primary and secondary series vaccines may be selected from—

| | Primary series | | Secondary series | |
|---|---|---|---|---|
| 1 | recombinant | recombinant | recombinant | recombinant |
| | adenovectored | adenovectored | adenovectored | adenovectored |
| | SARS-CoV-2 virus | SARS-CoV-2 virus | SARS-CoV-2 virus | SARS-CoV-2 virus |
| | vaccine. | vaccine. | vaccine. | vaccine. |
| 2 | killed-inactivated | killed-inactivated | killed-inactivated | killed-inactivated |
| | SARS-CoV-2 whole- | SARS-CoV-2 whole- | SARS-CoV-2 whole- | SARS-CoV-2 whole- |
| | virion vaccine | virion vaccine | virion vaccine | virion vaccine |
| 3 | killed-inactivated | killed-inactivated | recombinant | recombinant |
| | SARS-CoV-2 whole- | SARS-CoV-2 whole- | adenovectored | adenovectored |
| | virion vaccine | virion vaccine | SARS-CoV-2 virus | SARS-CoV-2 virus |
| | | | vaccine. | vaccine. |
| 4 | killed-inactivated | recombinant | killed-inactivated | recombinant |
| | SARS-CoV-2 whole- | adenovectored | SARS-CoV-2 whole- | adenovectored |
| | virion vaccine | SARS-CoV-2 virus | virion vaccine | SARS-CoV-2 virus |
| | | vaccine. | | vaccine. |
| 5 | recombinant human | recombinant human | recombinant human | recombinant human |
| | adenovirus (hAd- | adenovirus (hAd- | adenovirus (hAd- | adenovirus (hAd- |
| | SARS-CoV-2-S) | SARS-CoV-2-S) | SARS-CoV-2-S) | SARS-CoV-2-S) |
| 6 | recombinant | recombinant | recombinant | recombinant |
| | chimpanzee | chimpanzee | chimpanzee | chimpanzee |
| | adenovirus (ChAd- | adenovirus (ChAd- | adenovirus (ChAd- | adenovirus (ChAd- |
| | SARS-CoV-2-S) | SARS-CoV-2-S) | SARS-CoV-2-S) | SARS-CoV-2-S) |
| 7 | recombinant human | recombinant | recombinant human | recombinant |
| | adenovirus (hAd- | chimpanzee | adenovirus (hAd- | chimpanzee |
| | SARS-CoV-2-S) | adenovirus (ChAd- | SARS-CoV-2-S) | adenovirus (ChAd- |
| | | SARS-CoV-2-S) | | SARS-CoV-2-S) |
| 8 | recombinant human | recombinant human | recombinant | recombinant |
| | adenovirus (hAd- | adenovirus (hAd- | chimpanzee | chimpanzee |
| | SARS-CoV-2-S) | SARS-CoV-2-S) | adenovirus (ChAd- | adenovirus (ChAd- |
| | | | SARS-CoV-2-S) | SARS-CoV-2-S) |
| 9 | recombinant | recombinant | recombinant human | recombinant human |
| | chimpanzee | chimpanzee | adenovirus (hAd- | adenovirus (hAd- |
| | adenovirus (ChAd- | adenovirus (ChAd- | SARS-CoV-2-S) | SARS-CoV-2-S) |
| | SARS-CoV-2-S) | SARS-CoV-2-S) | | |
| 10 | killed-inactivated | killed-inactivated | recombinant human | recombinant human |
| | SARS-CoV-2 whole- | SARS-CoV-2 whole- | adenovirus (hAd- | adenovirus (hAd- |
| | virion vaccine | virion vaccine | SARS-CoV-2-S) | SARS-CoV-2-S) |
| 11 | killed-inactivated | killed-inactivated | recombinant | recombinant |
| | SARS-CoV-2 whole- | SARS-CoV-2 whole- | chimpanzee | chimpanzee |
| | virion vaccine | virion vaccine | adenovirus (ChAd- | adenovirus (ChAd- |
| | | | SARS-CoV-2-S) | SARS-CoV-2-S) |
| 12 | recombinant human | recombinant human | killed-inactivated | recombinant |
| | adenovirus (hAd- | adenovirus (hAd- | SARS-CoV-2 whole- | chimpanzee |
| | SARS-CoV-2-S) | SARS-CoV-2-S) | virion vaccine | adenovirus (ChAd- |
| | | | | SARS-CoV-2-S) |
| 13 | killed-inactivated | recombinant | killed-inactivated | recombinant |
| | SARS-CoV-2 whole- | chimpanzee | SARS-CoV-2 whole- | chimpanzee |
| | virion vaccine | adenovirus (ChAd- | virion vaccine | adenovirus (ChAd- |
| | | SARS-CoV-2-S) | | SARS-CoV-2-S) |
| 14 | killed-inactivated | killed-inactivated | recombinant | recombinant |
| | SARS-CoV-2 whole- | SARS-CoV-2 whole- | chimpanzee | chimpanzee |
| | virion vaccine | virion vaccine | adenovirus (ChAd- | adenovirus (ChAd- |
| | | | SARS-CoV-2-S) | SARS-CoV-2-S) |
| 15 | killed-inactivated | recombinant human | killed-inactivated | recombinant |
| | SARS-CoV-2 whole- | adenovirus (hAd- | SARS-CoV-2 whole- | chimpanzee |
| | virion vaccine | SARS-CoV-2-S) | virion vaccine | adenovirus (ChAd- |
| | | | | SARS-CoV-2-S) |

67. The method as claimed in claim 1, wherein, homologous route of administration comprises:

| Primary Series | Secondary Series |
|---|---|
| IM/IM | IM |
| IN/IN | IN |

68. The method as claimed in claim 1, wherein heterologous route comprises:

| Primary Series | Secondary Series |
|---|---|
| IM/IN | |
| IN/IM | |
| IM/IM | IN |
| IM/IM | IM/IN |
| IN/IN | IM |
| IM/IN | IN |
| IN/IM | IN |
